(19) United States
(12) Patent Application Publication
(10) Pub. No.: US 2014/0200501 A1
(43) Pub. Date: Jul. 17, 2014

(54) METHOD AND APPARATUS FOR SUTURELESSLY CONNECTING A CONDUIT TO A HOLLOW ORGAN

(71) Applicant: Correx, Inc., Waltham, MA (US)

(72) Inventors: Richard M. Beane, Hingham, MA (US); James Alan Crunkleton, Weston, MA (US); Anthony G. Liepert, Lincoln, MA (US); Joseph L. Smith, JR., Concord, MA (US)

(73) Assignee: Correx, Inc., Waltham, MA (US)

(21) Appl. No.: 13/967,826

(22) Filed: Aug. 15, 2013

Related U.S. Application Data
(63) Continuation of application No. 12/781,619, filed on May 17, 2010, now abandoned.
Application No. 12/781,619 is a continuation-in-part of application No. 11/783,287, filed on Apr. 6, 2007, now abandoned.
(60) Provisional application No. 60/789,563, filed on Apr. 6, 2006.
(60) Provisional application No. 60/821,019, filed on Aug. 1, 2006.
(60) Provisional application No. 61/226,374, filed on Jul. 17, 2009.
(60) Provisional application No. 61/304,979, filed on Feb. 16, 2010.

(51) Int. Cl. A61F 2/06; A61B 17/11

(52) U.S. Cl. CPC A61F 2/064; A61B 17/11
USPC 604/8; 606/155

(57) ABSTRACT

An implantable connector for suturelessly connecting a conduit to a hollow organ, the implantable connector comprising: a hollow expandable stent, wherein the hollow expandable stent comprises an internal skeleton and a blood-retaining membrane covering the internal skeleton, and wherein the hollow expandable stent is constructed so that it is capable of assuming (i) a diametrically-reduced state for insertion into an opening formed in the side wall of the hollow organ in order to create a first interference fit therewith, and (ii) a diametrically-expanded state for expanding against the side wall of the hollow organ in order to create a second, enhanced interference fit therewith.

### REFERENCE TO PENDING PRIOR PATENT APPLICATIONS

This patent application:

(i) is a continuation-in-part of pending prior U.S. patent application Ser. No. 11/783,287, filed Apr. 6, 2007 by Richard M. Beane et al. for APPARATUS AND METHOD FOR SUTURELESSLY CONNECTING A CONDUIT TO A HOLLOW ORGAN (Attorney's Docket No. CORREX-033058-000018), which in turn claims benefit of (a) prior U.S. Provisional Patent Application Ser. No. 60/789,563, filed Apr. 6, 2006 by Richard M. Beane et al. for APPARATUS AND METHOD FOR SUTURELESSLY CONNECTING A CONDUIT TO A HOLLOW ORGAN (Attorney's Docket No. CORREX-033058-0000011 PROV); and (b) prior U.S. Provisional Patent Application Ser. No. 60/821,019, filed Aug. 1, 2006 by Richard M. Beane et al. for APPARATUS AND METHOD FOR SUTURELESSLY CONNECTING A CONDUIT TO A HOLLOW ORGAN (Attorney's Docket No. CORREX-033058-0000012 PROV);

(ii) claims benefit of pending prior U.S. Provisional Patent Application Ser. No. 61/226,374, filed Jul. 17, 2009 by Richard M. Beane et al. for APPARATUS AND METHOD FOR SUTURELESSLY CONNECTING A CONDUIT TO A HOLLOW ORGAN (Attorney's Docket No. CORREX-44 PROV); and

(iii) claims benefit of pending prior U.S. Provisional Patent Application Ser. No. 61/304,979, filed Feb. 16, 2010 by Richard M. Beane et al. for APPARATUS AND METHOD FOR SUTURELESSLY CONNECTING A CONDUIT TO A HOLLOW ORGAN (Attorney's Docket No. CORREX-42A PROV).

The five (5) above-identified patent applications are hereby incorporated herein by reference.

### FIELD OF THE INVENTION

This invention relates to surgical methods and apparatus in general, and more particularly to surgical methods and apparatus for connecting a conduit to a hollow organ, and even more particularly to surgical methods and apparatus for connecting a vascular bypass conduit to the apex of the heart.

### BACKGROUND OF THE INVENTION

As the average age of the United States population increases, so do the instances of aortic stenosis.

Where the aortic stenosis is severe, the traditional treatment is the surgical replacement of the stenotic aortic valve via a conventional “open heart” procedure. However, this is a highly invasive approach, since it involves opening the patient's chest, establishing cardiopulmonary bypass with a so-called “heart-lung” machine, and then surgically opening the ascending aorta so as to access and replace the stenotic aortic valve. This approach typically presents substantial risk to the patient, particularly where the patient is elderly and/or otherwise in poor health.

An alternative approach to the conventional surgical replacement of the stenotic aortic valve involves the use of an apicoaortic conduit. In this approach, the native aortic valve is left in place, and a prosthetic valve is implanted in a parallel flow arrangement. More particularly, a vascular bypass conduit (or tube) is connected between the apex of the heart and the descending aorta. Somewhere along this apicoaortic conduit, the prosthetic valve is interposed. Thus, blood leaves the heart through the apex and travels through the apicoaortic conduit (with valve) to the descending aorta (see FIG. 1).

The traditional procedure for implanting an apicoaortic conduit is as follows.

First, the patient is placed on the operating table in the supine position. Anesthesia is induced, and the patient is intubated with a double-lumen endotracheal tube, which facilitates one-lung ventilation and allows the surgeon to work within the left chest. The patient is positioned with the left side up (i.e., turned approximately 90 degrees to the horizontal) and the pelvis is then rotated about 45 degrees, such that the femoral vessels are accessible.

Next, an incision is made over the femoral vessels, and the femoral artery and femoral vein are dissected out. Heparin is administered. Purse string sutures are placed in the femoral artery and in the femoral vein. Then the femoral artery is cannulated. First a needle is inserted into the femoral artery, and then a guidewire is inserted through the needle and into the femoral artery. Then the guidewire is advanced through the vascular system of the patient until the guidewire is located in the descending aorta. Transesophageal echo is used to ascertain that the guidewire is in the descending aorta. Once this is confirmed, an arterial cannula is inserted over the guidewire and into the artery using the Seldinger technique (Sven-Ivar Seldinger: Catheter replacement of the needle in percutaneous arteriography (a new technique), Acta Radiologica, Stockholm, 1953, 39:368-376). The arterial cannula is typically 19 French or 21 French. Once the arterial cannula has been inserted, purse string sutures are snugged down over tourniquets. A similar procedure is then followed to cannulate the femoral vein. The venous cannula is usually a few French larger than the arterial cannula. Once both the femoral artery and the femoral vein have been cannulated, the cannulae are connected to cardiopulmonary bypass (i.e., a heart-lung machine, etc.), so that the capability to initiate cardiopulmonary bypass at any time is present.

Next, a 1 cm incision is made in approximately the 6th interspace in the posterior auxiliary line, a videoscope (10 mm diameter) is inserted through the incision, and then the contents of the left chest are viewed. The location of the apex of the heart is determined, and the light from the videoscope is used to transilluminate the chest wall, which allows precise localization of the primary chest wall incision, which is to be made next. The primary chest wall incision is then performed, which is essentially an anterior thoracotomy, typically in the 6th interspace. Recent primary chest wall incisions have been about 10 cm long, but these incisions are expected to become smaller and smaller with time. A retractor is then inserted into the primary chest wall incision and the wound gently opened. A lung retractor is used to move the (deflated) left lung cephalad. A pledgeted suture is placed on the dome of the diaphragm and positioned so as to pull the diaphragm toward the feet (i.e., out of the way). The pericardium is then incised about the apex of the heart, and the apex is freed up and clearly identified.

At this point, the patient is ready to have the apicoaortic conduit connected to the apex of the heart and to the descending aorta.

The apicoaortic conduit is typically connected to the descending aorta first. The apicoaortic conduit is brought to the surgical field, and a measurement made from the apex of the heart to the descending aorta. The apicoaortic conduit is then trimmed appropriately. Next, a partial-occluding clamp is placed on the descending aorta, and the descending aorta is carefully opened with a knife and scissors. The outflow end of the apicoaortic conduit is then sutured to the descending aorta using 4-0 prolene suture, in a running stitch fashion. Once this has been completed, the clamp is removed and the anastomosis checked for hemostasis. Blood is contained by the presence of the prosthetic valve located within the apicoaortic conduit.

Next, the apicoaortic conduit is connected to the apex of the heart. This is traditionally the most technically challenging aspect of implanting the apicoaortic conduit. More particularly, connecting the apicoaortic conduit to the apex of the heart has been historically performed in a two-step process, by first cutting and removing a cylindrical plug of tissue from the apex of the heart, and then inserting the apicoaortic conduit into the formed hole and securing it in place. This two-step process creates the potential for significant blood loss after the hole has been formed in the wall of the heart and before the apicoaortic conduit is inserted into the formed hole and secured in place.

More particularly, the apicoaortic conduit has traditionally been connected to the apex of the heart in the following manner. First, the apicoaortic conduit is placed on the apex of the heart, and a marker is used to trace a circular outline of the apicoaortic conduit on the apex, in the planned location of insertion. Four large pledgeted sutures (i.e., mattress sutures) of 2-0 prolene are placed in the apex tissue, one in each quadrant surrounding the marked circle. The sutures are then brought through a sewing ring provided on the apicoaortic conduit. A stab wound is made in the apex of the heart (i.e., in the center of the traced circle), and a tonsil clamp is used to poke a hole into the left ventricle. Cardiopulmonary bypass is typically initiated at this point. A Foley catheter is then inserted into the left ventricle, and its balloon is expanded. Next, a cork borer is used to cut out a plug of tissue from the apex of the heart. This forms the hole which is to receive the apicoaortic conduit. The apicoaortic conduit is then parachuted down into position using the four pledgeted sutures. A rotary motion is generally necessary to seat the apicoaortic conduit in the formed hole in the apex. The four quadrant sutures are then tied, and hemostasis is checked. If there is a concern regarding hemostasis, additional sutures are placed.

With the apicoaortic conduit in place, cardiac function is then restored, with the apicoaortic conduit providing an alternative flow path between the left ventricle of the heart and the descending aorta, and with the prosthetic valve (located in the apicoaortic conduit) serving the same function as the aortic valve. The retractor is then removed, chest tubes are placed, and the wound is closed.

An alternative, improved method and apparatus for implanting the apicoaortic conduit is disclosed in U.S. patent application Ser. No. 11/086,577, filed Mar. 23, 2005 by Richard M. Beane et al. for APPARATUS AND METHOD FOR CONNECTING A CONDUIT TO A HOLLOW ORGAN (Attorney's Docket No. CORREX-033058-000005); Ser. No. 11/581,081, filed Oct. 16, 2006 by Richard M. Beane et al. for APPARATUS AND METHOD FOR FORMING A HOLE IN A HOLLOW ORGAN (Attorney's Docket No. CORREX-033058-000014); Ser. No. 11/783,287, filed Apr. 6, 2007 by Richard M. Beane et al. for APPARATUS AND METHOD FOR SUTURELESSLY CONNECTING A CONDUIT TO A HOLLOW ORGAN (Attorney's Docket No. CORREX-033058-000018); and Ser. No. 12/238,406, filed Sep. 25, 2008 by Richard M. Beane et al. for APPLICATOR, ASSEMBLY, AND METHOD FOR CONNECTING AN INLET CONDUIT TO A HOLLOW ORGAN (Attorney's Docket No. CORREX-033058-000036), which patent applications are hereby incorporated herein by reference. The aforementioned U.S. patent application Ser. Nos. 11/086,577, 11/581,081, 11/783,287 and 12/238,406 describe a novel system comprising an apicoaortic conduit and an applicator for implanting the apicoaortic conduit, with the applicator being adapted to cut and remove a cylindrical plug of tissue from the apex of the heart while simultaneously inserting the apicoaortic conduit into the formed hole. This novel system allows for placement of the apicoaortic conduit into the wall of the heart with minimal blood loss, so that cardiopulmonary bypass is not required. This is a major advance in the art.

More particularly, the aforementioned U.S. patent application Ser. Nos. 11/086,577, 11/581,081, 11/783,287 and 12/238,406 disclose, among other things, an apicoaortic conduit which comprises two parts, i.e., a left ventricle (LV) connector for connection to the apex of the heart, and a descending aorta connector (which contains the prosthetic valve) for connection to the descending aorta. With this new system, the descending aorta connector is preferably attached to the descending aorta first, then the LV connector is attached to the apex of the heart, and finally the LV connector is attached to the descending aorta connector, whereupon the apicoaortic conduit provides an alternative flow path (with valve) between the left ventricle of the heart and the descending aorta.

The new system disclosed in the aforementioned U.S. patent application Ser. Nos. 11/086,577, 11/581,081, 11/783,287 and 12/238,406 also includes an applicator for attaching the LV connector to the apex of the heart without the need for cardiopulmonary bypass. More particularly, the applicator comprises a pushing component, a coring component, and an expansion/retractor component. The coring component is mounted to the pushing component and carries the LV connector thereon. The expansion/retractor component is slidably coupled to the coring component, and is adapted to be passed through the apical wall of the left ventricle and then expanded. The expansion/retractor component seats against the inside apical wall of the left ventricle and provides support as the coring component is advanced through the myocardium, thereby enabling a clean tissue plug to be cut from the side wall of the heart while simultaneously implanting the LV connector in the apical wall of the heart, enscribing the cut tissue plug. The expansion/retractor component is then retracted into the coring component while the expansion/retractor component remains seated against the cut tissue plug, thereby carrying the cut tissue plug into the coring component. The LV connector is then sutured to the apical wall of the heart using sutures previously placed in the apical wall and a sewing ring provided on the LV connector.

The foregoing system is a major advance in the art, since it permits the LV connector to be implanted in the apical wall of the heart with minimal blood loss, so that cardiopulmonary bypass is not required.

However, even using the improved method and apparatus of the aforementioned U.S. patent application Ser. Nos. 11/086,577, 11/581,081, 11/783,287 and 12/238,406, successfully implanting the LV connector into the apex of the heart remains a challenging aspect of the apicoaortic bypass procedure. This is because of the need to place deep, near-full-thickness sutures into the wall of the heart (in order to avoid pseudoaneurysms), in addition to the need to use pledgeted, mattress sutures (in order to avoid “pull-through” in friable heart tissue), both of which make the procedure of securing the LV connector to the wall of the heart both technically challenging and time-consuming. See, for example, FIG. 2, which illustrates the near-full-thickness suture placement required for proper hemostasis when suturing an LV connector to the wall of the heart.

Some references that discuss the requirements for successful implantation of an apicoaortic conduit are listed below:

(i) Aortic Valve Bypass Surgery: Midterm Clinical Outcomes in a High-Risk Aortic Stenosis Population, James S. Gammie, MD, Leandra S. Krowsoski BA, James M. Brown, MD, Patrick N. Odonkor, MD, Cindi A. Young, Mary J. Santos, PA-C, John S. Gottdiener, MD, and Bartley P. Griffith, Circulation 2008; 118:1460-1466. http://circ.ahajournals.org/cgi/content/short/118/14/1 460

(ii) Aortic Valve Bypass for the High-Risk Patient with Aortic Stenosis, James S. Gammie, MD, John W. Brown, MD, The Annals of Thoracic Surgery, 2006, http://ats.ctsnetjournals.org/cgi/content/abstract/81/5/1605

(iii) Aortic Valve Bypass for aortic stenosis: imaging appearances on multidetector CT, Charles White, . . . , James S. Gammie, MD, The International Journal of Cardiovascular Imaging, Jul. 20, 2006. http://www.springerlink.com/content/am767116081052p6/

(iv) Hemodynamic Efficacy of the Aortic Valve Bypass (Apicoaortic Conduit): Assessment by 2D-Doppler Echocardiography, James S. Gammie, Bartley P. Griffith, Jamie M. Brown, Mary J. Santos, Karen Roberts, Patrick N. Odonkor, John S. Gottdiener. Division of Cardiac Surgery, University of Maryland Medical Center, Baltimore, Md., USA, ISMICS Annual Scientific Meeting, 2006. http://www.ismics.org/abstracts/2006/MP9.html

(v) Heart Valve Disease: Achievements and Challenges, James S. Gammie, MD, Elias Balares, PhD. http://www.enme.umd.edu/events/RRD/2007/Presentations/heartvalve/ResearchDay07Talk.pdf

(vi) Off-pump apicoaortic conduit insertion for high-risk patients with aortic stenosis, Thomas A. Vassiliades, Jr., MD, European Journal of Cardio-Thoracic Surgery, 2003. http://ejcts.ctsnetjournals.org/cgi/content/abstract/2 3/2/156

Some prior art, in attempting to develop connector devices that implant in the heart wall, assumes a smooth heart wall of constant thickness and operates by sandwiching tissue between opposing parallel plates. See, for example, FIG. 12B of U.S. patent application Ser. No. 11/770,288, filed Jun. 28, 2007 by William E. Cohn for AUTOMATED SURGICAL CONNECTOR, and FIGS. 8A and 8B of U.S. patent application Ser. No. 11/251,100, filed Oct. 14, 2005 by Thomas Vassiliades et al. for VASCULAR CONDUIT DEVICE AND SYSTEM FOR IMPLANTING, which two patent applications are hereby incorporated herein by reference. In reality, however, the interior of the left ventricle of the heart is generally not a smooth continuous surface, and the wall thickness of the left ventricle generally varies considerably within any given patient, and also from patient to patient. As a result, the methods and apparatus disclosed in the aforementioned U.S. patent application Ser. Nos. 11/770,288 and 11/251,100 can present issues when applied in actual patient anatomies.

Consequently, a new and improved approach is needed for connecting an implantable connector to a hollow organ, and particularly for connecting an apicoaortic conduit to the apex of the heart.

The present invention addresses the aforementioned difficulties associated with connecting an implantable connector to a hollow organ, and particularly with connecting an apicoaortic conduit to the apex of the heart, by providing new enabling technology, surgical tools and procedures to achieve a sutureless connection, and particularly a sutureless apical connection.

### SUMMARY OF THE INVENTION

The purpose of this invention is to enable sutureless placement of an implantable connector, preferably an LV connector of an apicoaortic conduit, into the wall of a hollow organ, preferably the side wall of the left ventricle of the heart, and preferably using an applicator of the sort disclosed in the aforementioned U.S. patent application Ser. Nos. 11/086,577, 11/581,081, 11/783,287 and 12/238,406. This implantable connector is intended to facilitate automatic placement, and sutureless securement, of the implantable connector in the tissue wall with minimal blood loss.

The implantable connector may consist in part of a hollow expandable stent, wherein the hollow expandable stent comprises an internal skeleton covered with a blood-retaining membrane (e.g., fabric). The hollow expandable stent is capable of assuming (i) a diametrically-reduced state, and (ii) a diametrically-expanded state. The implantable connector is inserted into the hole formed in the hollow organ (e.g., the apical wall) while the hollow expandable stent is in its diametrically-reduced state, and then the hollow expandable stent is reconfigured into its diametrically-expanded state so as to secure the implantable connector in the formed hole, whereby to effect sutureless securement of the implantable connector in the tissue wall.

In one preferred form of the invention, the internal skeleton of the hollow expandable stent comprises an internal spring. The internal spring is normally in an axially-contracted state, but is capable of being stretched axially. The blood-retaining membrane is applied to the internal spring while the internal spring is in its axially-stretched state, so that the blood-retaining membrane gathers (and projects radially) when the internal spring is in its normal, axially-contracted state. In this form of the invention, the implantable connector, normally in an axially-contracted state, is stretched axially, and held in this axially-stretched condition, for implantation into a hole formed in the hollow organ (e.g., a cored hole formed in the apical wall of the heart). Once implanted, the implantable connector is allowed to axially contract, which causes the blood-retaining membrane covering the internal spring to gather, thereby increasing the diameter of the implantable connector and locking the implantable connector in the formed hole.

In one preferred form of the invention, the internal spring comprises a cylindrical spring. This cylindrical spring may be a coiled spring or an equivalent structure. A coiled spring (or an equivalent structure) can be advantageous since it tends to increase in diameter as it changes from an axially-stretched condition to an axially-contracted condition. This increase in diameter helps bind the implantable connector in the formed hole in the tissue, and acts in addition to the binder already being provided by the gathering blood-retaining membrane. The implantable connector preferably also includes a flange disposed on the outer surface of the hollow expandable stent, intermediate its length, for tightly engaging against the outer surface of the tissue.

In another preferred form of the invention, the hollow expandable stent comprises a frusto-conical structure, with the wider end of the frusto-conical structure leading and with the narrower end of the frusto-conical structure trailing, and with the frusto-conical structure being capable of assuming (i) a diametrically-reduced state, and (ii) a diametrically-expanded state. The implantable connector preferably also includes a flange disposed on the outer surface of the implantable connector, intermediate its length, for engaging against the outer surface of the tissue into which the implantable connector is to be deployed. As a result of this construction, by inserting the implantable connector into a hole in the tissue while the frusto-conical structure is in its diametrically-reduced state, and thereafter reconfiguring the frusto-conical structure into its diametrically-expanded state, the implantable connector engages the side wall of the formed hole, thereby suturelessly securing the implantable connector in the tissue wall. Significantly, the frusto-conical structure exerts a compressive force on the host tissue as the wider end of the frusto-conical structure and the flange of the implantable connector are brought together.

In another form of the invention, the frusto-conical structure comprises a frusto-conical coiled spring, with the wider end of the frusto-conical spring leading and with the narrower end of the frusto-conical spring trailing, and the implantable connector preferably includes a flange disposed on the outer surface of the implantable connector, intermediate its length, for engaging against the outer surface of the tissue into which the implantable connector is to be deployed. As a result of this construction, by axially extending and torsionally stretching the frusto-conical spring so that it assumes a generally cylindrical configuration and so that the implantable connector assumes a diametrically-reduced configuration, inserting the implantable connector into a hole in the tissue while the implantable connector is in its diametrically-reduced state, and thereafter releasing the frusto-conical spring so that it axially contracts and torsionally unwinds so that the spring returns to its frusto-conical configuration and the implantable connector assumes its diametrically-expanded state, the frusto-conical spring exerts a compressive force on the host tissue, as the wider end of the frusto-conical spring and the flange of the implantable connector are brought together.

In one preferred form of the present invention, the frusto-conical structure comprises a Z-stent.

In another preferred form of the present invention, the frusto-conical structure comprises a plurality of torsional springs.

In still another preferred form of the present invention, the frusto-conical structure comprises interwoven wire springs.

In yet another preferred form of the present invention, the frusto-conical structure comprises a plurality of telescoping members.

In another preferred form of the present invention, the frusto-conical structure comprises a generally cylindrical structure including hinges intermediate its length. In still another preferred form of the present invention, the frusto-conical structure comprises a plurality of cantilevered fingers.

In addition, bioglue can also be used to enhance the sealing effect of the flange against the outer wall of the tissue, thereby helping to ensure hemostasis.

In one preferred form of the invention, there is provided an implantable connector for suturelessly connecting a conduit to a hollow organ, the implantable connector comprising:

a hollow expandable stent, wherein the hollow expandable stent comprises an internal skeleton and a blood-retaining membrane covering the internal skeleton, and wherein the hollow expandable stent is constructed so that it is capable of assuming (i) a diametrically-reduced state for insertion into an opening formed in the side wall of the hollow organ in order to create a first interference fit therewith, and (ii) a diametrically-expanded state for expanding against the side wall of the hollow organ in order to create a second, enhanced interference fit therewith.

In another preferred form of the present invention, there is provided a system for suturelessly connecting a conduit to a hollow organ, the system comprising:

an applicator comprising a pushing component, a coring component, and an expansion/retractor component, the coring component being mounted to the pushing component, and the expansion/retractor component being slidably coupled to the coring component and adapted to pass through a side wall of the hollow organ; and

an implantable connector mounted to the coring component of the applicator, the implantable connector comprising:
a hollow expandable stent, wherein the hollow expandable stent comprises an internal skeleton and a blood-retaining membrane covering the internal skeleton, and wherein the hollow expandable stent is constructed so that it is capable of assuming (i) a diametrically-reduced state closely sized to the coring component for insertion into an opening formed in the side wall of the hollow organ in order to create a first interference fit therewith, and (ii) a diametrically-expanded state substantially larger than the coring component for expanding against the side wall of the hollow organ in order to create a second, enhanced interference fit therewith.

In another preferred form of the present invention, there is provided a method for suturelessly connecting a conduit to a hollow organ, the method comprising the steps of:

mounting an implantable connector to a coring component;

forming an opening in the side wall of the hollow organ by advancing the coring component with respect to the side wall of the hollow organ, with the implantable connector being carried into the opening formed by the coring component;

diametrically expanding the implantable connector within the formed opening so as to secure the implantable connector to the side wall of the hollow organ, and removing the coring component from the formed opening.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a conventional apicoaortic conduit.

FIG. 2 is a schematic view showing how near-full-thickness sutures are currently used to secure a conventional apicoaortic conduit to the apex of the heart.

FIG. 3 is a schematic view showing a first left ventricle (LV) connector formed in accordance with the present invention, wherein the hollow expandable stent of the LV connector comprises a cylindrical coiled spring, and further wherein the cylindrical coiled spring is shown in its axially-contracted state.

FIG. 4 is a schematic view showing the LV connector of FIG. 3, but with the cylindrical coiled spring being shown in its axially-expanded (i.e., stretched) condition.

FIG. 5 is a schematic perspective, sectional view showing the LV connector of FIG. 3, but with the portion of the cylindrical coiled spring distal to the flange being shown in its axially-expanded (i.e., stretched) condition and with the portion of the cylindrical coiled spring proximal to the flange being shown in its straight condition.

FIG. 6 is a schematic perspective, sectional view showing the LV connector of FIG. 3, but with the portion of the cylindrical coiled spring distal to the flange being shown in its axially-contracted condition and with the portion of the cylindrical coiled spring proximal to the flange being shown in a bent condition.

FIG. 7 is a schematic view showing the LV connector of FIG. 3 in partial section and with its cylindrical coiled spring in its axially-expanded (i.e., stretched) condition, and showing an applicator which may be used to insert the LV connector into the side wall of the apex of the heart, such as the applicator disclosed in the aforementioned U.S. patent application Ser. Nos. 11/086,577, 11/581,081, 11/783,287 and 12/238,406.

FIG. 8 is a schematic view, partially in section, showing the LV connector of FIG. 3 inserted into the left ventricle of the heart, wherein the portion of the cylindrical coiled spring distal to the flange is in its axially-expanded state, so that the hollow expandable stent is in its diametrically-reduced state.

FIG. 9 is a schematic view, partially in section, showing the LV connector of FIG. 3 inserted into the left ventricle of the heart, wherein the portion of the cylindrical coiled spring distal to the flange is in its axially-contracted state, so that the hollow expandable stent is in its diametrically expanded state.

FIG. 10 is a schematic view, partially in section, of a second LV connector formed in accordance with the present invention, wherein the hollow expandable stent of the LV connector comprises a cylindrical coiled spring, and further wherein the portion of the cylindrical coiled spring distal to the flange is in its axially-extended (i.e., stretched) condition. In this form of the invention, the distal end of the LV connector also comprises radially-deployable elements, with the radially-deployable elements being shown in FIG. 10 in their radially-retracted position.

FIG. 11 is a schematic view, partially in section, showing the LV connector of FIG. 10, but with the portion of the cylindrical coiled spring distal to the flange being in its axially-contracted condition, and with the radially-deployable elements on the distal end of the LV connector being in their radially-extended position.

FIG. 12 is a schematic view, partially in section, showing a third LV connector formed in accordance with the present invention, wherein the hollow expandable stent of the LV connector comprises a cylindrical coiled spring, and further wherein the portion of the cylindrical coiled spring distal to the flange is in its axially-expanded (i.e., stretched) condition. In this form of the invention, the distal end of the LV connector also comprises radially-deployable finger elements, with the radially-deployable finger elements being shown in FIG. 12 in their radially-retracted position.

FIG. 13 is a schematic view, partially in section, showing the LV connector of FIG. 12, but with the portion of the cylindrical coiled spring distal to the flange being in its axially-expanded (i.e., stretched) condition, and with the radially-deployable finger elements on the distal end of the LV connector being in their radially-extended position.

FIG. 14 is a schematic view, partially in section, showing the LV connector of FIG. 12, but with the portion of the cylindrical coiled spring distal to the flange being in its axially-contracted condition, and with the radially-deployable finger elements on the distal end of the LV connector being in their radially-extended position.

FIG. 15 is a schematic view, partially in section, showing a fourth LV connector formed in accordance with the present invention, wherein the hollow expandable stent of the LV connector comprises a cylindrical coiled spring, and further wherein the portion of the cylindrical coiled spring distal to the flange is in its axially-expanded (i.e., stretched) condition. In this form of the invention, the distal end of the LV connector also comprises a radially-deployable balloon element, with the radially-deployable balloon element being shown in FIG. 15 in a folded and retracted position.

FIG. 16 is a schematic view, partially in section, showing the LV connector of FIG. 15, but with the portion of the cylindrical coiled spring distal to the flange in its axially-expanded (i.e., stretched) condition, and with the radially-deployable balloon element on the distal end of the LV connector in its inflated position.

FIG. 17 is a schematic view, partially in section, showing the LV connector of FIG. 15, but with the portion of the cylindrical coiled spring distal to the flange in its axially-contracted condition, and with the radially-deployable balloon element in its inflated position.

FIG. 18 is a schematic view, partially in section, showing a fifth LV connector formed in accordance with the present invention, wherein the hollow expandable stent of the LV connector comprises a cylindrical coiled spring, and further wherein the portion of the cylindrical coiled spring distal to the flange is in its axially-expanded (i.e., stretched) condition. In this form of the invention, the distal end of the LV connector also comprises a radially-deployable torsional spring element, with the radially-deployable torsional spring element being shown in FIG. 18 in its radially-retracted position.

FIG. 19 is a schematic view, partially in section, showing the LV connector of FIG. 18, but with the radially-deployable torsional spring element disposed on the distal end of the LV connector being shown in its radially-deployed position.

FIG. 20 is a schematic view, partially in section, showing the LV connector of FIG. 18, but with the portion of the cylindrical coiled spring distal to the flange in its axially-contracted condition, and with the radially-deployable torsional spring element disposed on the distal end of the LV connector in its radially-deployed position.

FIG. 21 is a schematic view, partially in section, showing a sixth LV connector formed in accordance with the present invention, wherein the hollow expandable stent of the LV connector comprises a frusto-conical coiled spring, and further wherein the portion of the frusto-conical coiled spring distal to the flange is in its torsionally-contracted and axially-extended position, so that the frusto-conical coiled spring assumes a generally cylindrical configuration.

FIG. 22 is a schematic view, partially in section, showing the LV connector of FIG. 21, but with the torsional compression spring shown in its released condition, wherein it assumes a radial spiral shape and a compressively axially-contracted condition.

FIG. 23 is a schematic view, partially in section, showing a seventh LV connector formed in accordance with the present invention, wherein the hollow expandable stent of the LV connector comprises a frusto-conical structure, and further wherein the portion of the frusto-conical structure at the distal end of the hollow expandable stent is in its diametrically-reduced state. In this form of the invention, the frusto-conical structure preferably comprises a Z-stent. The hollow expandable stent is preferably constructed in a manner similar to an expanding stent graft, wherein the Z-stent comprises expanding units.

FIG. 24 is a schematic view, partially in section, showing the LV connector of FIG. 23, but with the portion of the frusto-conical structure at the distal end of the hollow expandable stent being in its diametrically-expanded state.

FIG. 25 is a schematic view, partially in section, showing an eighth LV connector formed in accordance with the present invention, wherein the hollow expandable stent of the LV connector comprises a frusto-conical structure, and further wherein the portion of the frusto-conical structure at the distal end of the hollow expandable stent is in its diametrically-reduced state. In this form of the invention, the frusto-conical structure comprises a plurality of torsional springs, with the torsional springs at the distal end of the frusto-conical structure being shown in FIG. 25 torsionally contracted, and held in this condition by a locking pin, so that the hollow expandable stent assumes a generally cylindrical configuration.

FIG. 26 is a schematic view, partially in section, showing the LV connector of FIG. 25, but with the torsional springs being shown in their released condition, wherein the LV connector assumes a diametrically-expanded conical shape.

FIG. 27 is a schematic view showing a single torsional spring (with locking pin) from the LV connector of FIG. 25, with the torsional spring being shown in both the contracted and expanded conditions.

FIG. 28 is a schematic view, partially in section, showing a ninth LV connector formed in accordance with the present invention, wherein the hollow expandable stent of the LV connector comprises a frusto-conical structure, and further wherein the portion of the frusto-conical structure at the distal end of the LV connector is shown in its diametrically-reduced state. In this form of the invention, the frusto-conical structure comprises two or more frusto-conical coiled springs, with the frusto-conical coiled springs being shown in FIG. 28 in their torsionally-contracted position, and being held in position with a retaining pin (such as a retaining pin of the sort shown in FIG. 25), so that the hollow expandable stent assumes a generally cylindrical configuration.

FIG. 29 is a schematic view showing the LV connector of FIG. 28, but with the frusto-conical coiled springs being shown in their released condition, wherein the LV connector assumes a diametrically-expanded conical shape.

FIG. 30 is a schematic view, partially in section, showing a tenth LV connector formed in accordance with the present invention, wherein the hollow expandable stent of the LV connector comprises a frusto-conical coiled spring, and further wherein the portion of the frusto-conical coiled spring at the distal end of the LV connector is in its diametrically-reduced state, so that the hollow expandable stent assumes a generally cylindrical configuration. In this form of the invention, inner and outer blood-retaining membranes are mounted to the frusto-conical coiled spring, with the inner and outer membranes being in a vertically pleated or folded condition when the frusto-conical coiled spring is in its diametrically-constrained condition, as shown in FIG. 30.

FIG. 31 is a schematic view showing the LV connector of FIG. 30, but with the frusto-conical coiled spring in its released condition, wherein the LV connector assumes a diametrically-expanded conical shape, and further wherein the inner and outer membranes bow inwardly between the coils of the spring, creating a ribbed effect for the hollow expandable stent.

FIG. 32 is a schematic view, partially in section, showing an eleventh LV connector formed in accordance with the present invention, wherein the hollow expandable stent of the LV connector comprises a cylindrical coiled spring, and further wherein the portion of the coiled spring distal to the flange is in its axially-expanded (i.e., stretched) condition. In this form of the invention, a tubular foam layer has been added to the outside of the outer blood-retaining membrane.

FIG. 33 is a schematic view showing the LV connector of FIG. 32, but with the cylindrical coiled spring in its released (i.e., axially-contracted) condition. The outer blood-retaining membrane and the tubular foam layer are shown in their gathered condition, so that the hollow expandable stent is in its diametrically-expanded state.

FIG. 34 is a schematic view, partially in section, showing a twelfth LV connector formed in accordance of the present invention, wherein the hollow expandable stent of the LV connector comprises a cylindrical coiled spring, and further wherein the portion of the cylindrical coiled spring distal to the flange is in its axially-expanded (i.e., stretched) state. In this form of the invention, a foam layer has been added between the inner and outer blood-retaining membranes.

FIG. 35 is a schematic view showing the LV connector of FIG. 34, but with the cylindrical coiled spring in its released (i.e., axially-contracted) condition, and with the inner and outer blood-retaining membranes, and the intermediate foam layer, being shown in their gathered condition, so that the hollow expandable stent is in its diametrically-expanded state.

FIG. 36 is a schematic view, partially in section, showing a thirteenth LV connector formed in accordance with the present invention, wherein the hollow expandable stent of the LV connector comprises a cylindrical coiled spring, and further wherein the portion of the cylindrical coiled spring distal to the flange is in its axially-expanded (i.e., stretched) condition. In this form of the invention, a foam washer has been provided between the flange and the outer surface of the heart wall.

FIG. 37 is a schematic view, partially in section, showing a fourteenth LV connector formed in accordance with the present invention, wherein the hollow expandable stent of the LV connector comprises a generally frusto-conical structure, and further wherein the portion of the generally frusto-conical structure at the distal end of the LV connector is in its diametrically-reduced state. In this form of the invention, the generally frusto-conical structure comprises a series of interwoven wire springs, with the interwoven wire springs being shown in FIG. 37 in their diametrically-reduced state, held in place by a stay suture and ripcord, so that the hollow expandable stent assumes a generally cylindrical configuration.

FIG. 38 is a schematic view showing the LV connector of FIG. 37, but with the interwoven wire springs being shown in their released position, wherein the LV connector resumes its normal bell or trumpet shape.

FIG. 39 is a schematic view, partially in section, showing a fifteenth LV connector formed in accordance with the present invention, wherein the hollow expandable stent of the LV connector comprises a frusto-conical coiled spring, and further wherein the portion of the frusto-conical coiled spring at the distal end of the LV connector is in its diametrically-reduced state, so that the hollow expandable stent assumes a generally cylindrical configuration. In this form of the invention, the frusto-conical coiled spring is held in its diametrically-reduced state by a tubular thin film element containing stop elements and tear strip tubular elements.

FIG. 40 is a schematic view showing the LV connector of FIG. 39, but with the frusto-conical coiled spring collapsing into its diametrically-expanded state, after the tear strip tubular elements are removed from around the LV connector.

FIG. 41 is a schematic view, partially in section, showing a sixteenth LV connector formed in accordance with the present invention, wherein the hollow expandable stent of the LV connector comprises a frusto-conical structure, and further wherein the portion of the frusto-conical structure at the distal end of the LV connector is in its diametrically-reduced state, so that the hollow expandable stent assumes a generally cylindrical configuration. In this form of the invention, the frusto-conical structure comprises a pair of telescoping members, wherein relative movement of the telescoping members towards one another cams the outer member radially outward so that the hollow expandable stent assumes a generally frusto-conical configuration, and relative movement of the telescoping members away from one another permits the outer member to return radially inward so that the hollow expandable stent assumes a generally cylindrical configuration.

FIG. 42 is a schematic view showing the LV connector of FIG. 41, but with relative movement of the telescoping members towards one another, causing the fingers of the outer member to be forced radially outward until latching elements engage, thereby locking the frusto-conical structure in a radially-expanded conical state.

FIGS. 43 and 44 are schematic views, partially in section, showing the latching mechanisms of the LV connector of FIG. 41.

FIG. 45 is a schematic view, partially in section, showing a seventeenth LV connector formed in accordance with the present invention, wherein the hollow expandable stent of the LV connector comprises a frusto-conical structure, and further wherein the portion of the frusto-conical structure at the distal end of the LV connector is in its diametrically-reduced state, so that the hollow expandable stent assumes a generally cylindrical configuration. In this form of the invention, the frusto-conical structure comprises a pair of coaxial members, wherein axial compression of the coaxial members causes the frusto-conical structure to expand radially outwardly so that the hollow expandable stent assumes a generally frusto-conical configuration, and axial tension of the coaxial members causes the frusto-conical structure to return radially inwardly, so that the hollow expandable stent assumes a generally cylindrical configuration.

FIG. 46 is a schematic view, partially in section, showing the LV connector of FIG. 45, but with the coaxial members axially compressed so that the fingers of the outer coaxial member bend at the hinge points, thereby buckling radially outwardly.

FIG. 47 is a schematic view, partially in section, showing an eighteenth LV connector formed in accordance with the present invention, wherein the hollow expandable stent of the LV connector comprises a frusto-conical structure, and further wherein the portion of the frusto-conical structure at the distal end of the LV connector is shown in its diametrically-reduced state so that the hollow expandable stent assumes a generally cylindrical configuration. In this form of the invention, the frusto-conical structure comprises a plurality of cantilevered fingers arranged in a circle, the fingers being attached at their flange end and free at their distal ends.

FIG. 48 is a schematic view, partially in section, showing the LV connector of FIG. 47, but with the frusto-conical structure being shown in its diametrically-expanded state, so that the hollow expandable stent assumes a generally frusto-conical configuration.

### DETAILED DESCRIPTION OF THE PREFERRED EMBODIMENTS

### The New LV Connector in General

The present invention provides for sutureless anastomosis between a conduit and a hollow organ, and preferably between an apicoaortic conduit and the left ventricle of the heart. Largely because of the previous need to place pledgeted, near-full-thickness mattress sutures through the wall of the heart, in sufficient number to prevent pull-out via the “cheese-cutting” effect of sutures, especially in older, more friable tissue, while simultaneously providing enough tension on the sutures to prevent blood leakage and the formation of pseudoaneurysms, this portion of an apicoaortic bypass procedure has traditionally been time-consuming and technically challenging when using conventional, suture-based approaches. Significantly, the present invention renders the anastomotic connection of the apicoaortic conduit to the apex of the heart relatively fast, technically less challenging and highly reliable. The present invention may also be used to attach other conduits to other hollow organs in a sutureless, fluid-tight connection.

In addition to the objectives already described, another object of the present invention is to allow the surgeon to place, and simultaneously deploy, securement mechanisms on the apicoartic conduit so as achieve complete hemostasis, with no pseudoaneurysms, and without requiring the use of pledgeted mattress sutures.

In a particular embodiment of the present invention, the apicoaortic conduit comprises a left ventricle (LV) connector for connection to the apex of the heart, and a descending aorta connector (which contains the prosthetic valve) for connection to the descending aorta, with the LV connector being joinable to the descending aorta connector so as to form the complete apicoaortic conduit.

The LV connector may consist in part of a hollow expandable stent, wherein the hollow expandable stent comprises an internal skeleton covered with a blood-retaining membrane, e.g., fabric. The hollow expandable stent is capable of assuming (i) a diametrically-reduced state, and (ii) a diametrically-expanded state. The implantable connector is inserted into the hole formed in the hollow organ (e.g., the apical wall) while the hollow expandable stent is in its diametrically-reduced state, and then the hollow expandable stent is reconfigured into its diametrically-expanded state so as to secure the implantable connector in the formed hole, whereby to effect sutureless securement of the implantable connector in the tissue wall.

In one preferred form of the invention, the internal skeleton of the hollow expandable stent comprises an internal spring. The internal spring is normally in an axially-contracted state, but is capable of being stretched axially. The blood-retaining membrane is applied to the internal spring while the internal spring is in its axially-stretched state. As a result, when the internal spring is thereafter allowed to contract, the blood-retaining membrane covering the internal spring collapses upon itself, producing a series of tight, pleat-like folds, each having an outside radius larger than the outside diameter of the blood-retaining membrane when the internal spring is in its axially-stretched condition, whereby to bind the LV connector in a hole formed in the apical wall of the heart.

In one preferred form of the invention, the internal spring is preferably also constructed so that the contracting spring increases in size radially as it decreases in size axially, thereby further contributing to the overall increase in the diameter of the implanted portion of the LV connector.

By way of example but not limitation, a cylindrical coiled spring may be used for the internal spring of the LV connector.

The LV connector preferably also includes a flange to bear against the outer surface of the heart.

Implantation of an LV connector of the sort disclosed above may be achieved using an applicator of the sort disclosed in the aforementioned U.S. patent application Ser. Nos. 11/086,577, 11/581,081, 11/783,287 and 12/238,406. Implantation using an applicator of this sort is preferred, since it allows the surgeon to core a hole through the apex of the heart while simultaneously implanting the LV connector. During implantation, the internal spring of the LV connector is preferably held in its axially-extended position by latching mechanisms on the applicator, or by a “pull pin” or “releasing suture” which may be activated independently of the applicator, as will hereinafter be discussed.

Implantation proceeds until the flange, preferably slightly dish-shaped (or cupped) disposed on the outer surface of the LV connector, comes into full contact with the epicardium of the heart. When full contact is established, the latching means on the applicator (or the “pull pin” or “releasing suture”) are released and the axially-expanded internal spring contracts axially toward the flange. As the internal spring contracts axially, the blood-retaining membrane covering the internal spring is forced to collapse into a series of tight folds. These tight folds cause the membrane to project radially outboard of its previous position, i.e., the position occupied when the internal spring was axially-expanded (i.e., stretched). In addition, the contracting internal spring preferably also expands radially within the formed hole, thereby further securing the LV connector to the tissue. Furthermore, using an applicator such as that described in the aforementioned U.S. patent application Ser. Nos. 11/086,577, 11/581,081, 11/783,287 and 12/238,406 automatically produces a hole in the wall of the heart which is smaller than the LV connector's outside diameter, because the coring component of the applicator resides within the LV connector during implantation. Thus, the axially collapsing and radially expanding LV connector puts additional radial force on the already-undersized hole formed in the heart.

The tight fit of the LV connector in the formed hole, and the additional radial expansion of the LV connector while within the formed hole, produces hemostasis and keeps the LV connector from popping out of the hole formed in the heart wall.

The torsional characteristics of the internal spring can improve retention in the heart wall if, when the axially-expanded internal spring is released, torsional force causes the internal spring to assume an axially shortened spiral shape, with the larger part of the spiral near the inner surface of the heart. In other words, if the internal spring of the LV connector comprises a frusto-conical coiled spring, the spring can be axially-stretched and torsionally-constrained so that it will assume a generally cylindrical configuration, so that the hollow expandable stent will have a diametrically-reduced configuration; however, upon release, the spring will reassume its original frusto-conical configuration, so that the hollow expandable stent will have a diametrically-expanded configuration. Significantly, with this form of the invention, a net inward force is created which tends to pull the LV connector into the heart. This tendency of the LV connector to move inwardly is checked by the presence of the flange, which is pressing against the epicardium, whereby to make the connection between the LV connector and the heart even more secure.

In addition to variations in the shape of the internal spring of the LV connector, various mechanisms and devices can be added to the distal end of the LV connector which, when deployed, occupy an area much larger than the cored hole in the wall of the heart. Thus, the LV connector cannot be forced back out of the left ventricle by blood pressure, muscle contractions or other forces that the heart can produce; in other words, the LV connector cannot “pop out” due to left ventricle (LV) pressure or heart wall motions.

The cup-shaped (or dish-shaped) flange, once in contact with the epicardium, cannot move further inwardly in response to the aforementioned spring forces generated by the internal spring of the LV connector, and so acts in counter-tension to that spring force, compressing the heart wall tissue between them. This squeezing pressure over the entire thickness of the heart wall helps provide hemostasis while preventing pseudoaneurysms.

The flange is preferably made out of a material similar to that used for a standard sewing ring, so that the new device retains the functionality of a conventional implantable connector (i.e., it may be sutured in place), should the need arise.

In one preferred form of the invention, the internal skeleton of the hollow expandable stent comprises a frusto-conical structure, with the wider end of the frusto-conical structure leading and with the narrower end of the frusto-conical structure trailing, and with the frusto-conical structure being capable of assuming (i) a diametrically-reduced state, and (ii) a diametrically-expanded state. The implantable connector preferably also includes a flange disposed on the outer surface of the implantable connector, intermediate its length, for engaging against the outer surface of the tissue. As a result of this construction, by inserting the implantable connector into a hole in the tissue while the frusto-conical structure is in its diametrically-reduced state, and thereafter reconfiguring the frusto-conical structure into its diametrically-expanded state, the implantable connector engages the side wall of the formed hole, thereby suturelessly securing the implantable connector in the tissue wall. In addition, the frusto-conical structure exerts a compressive force on the host tissue as the wider end of the frusto-conical structure and the flange of the implantable connector are brought together.

In one preferred form of the present invention, the frusto-conical structure comprises a frusto-conical coiled spring. In another preferred form of the present invention, the frusto-conical structure comprises a Z-stent. In another preferred form of the present invention, the frusto-conical structure comprises a plurality of torsional springs. In still another preferred form of the present invention, the frusto-conical structure comprises interwoven wire springs. In yet another preferred form of the present invention, the frusto-conical structure comprises a plurality of telescoping members. In another preferred form of the present invention, the frusto-conical structure comprises a generally cylindrical structure including hinges intermediate its length. In still another preferred form of the present invention, the frusto-conical structure comprises a plurality of cantilevered fingers.

If desired, various hemostatic agents and materials may be impregnated into, or attached to, or applied onto, the LV connector so as to aid in creating hemostasis, achieving a tighter fit and thus preventing pop-out, and/or to produce a more rapid coagulation cascade (and therefore a shorter time to tissue in-growth).

Thus, and as will hereinafter be discussed in further detail, the present invention provides a new and improved method and apparatus for effecting an anastomotic joinder between a conduit and a hollow organ, and preferably between an apicoaortic conduit and the apex of the heart, wherein the joinder may utilize radial expansion against the surrounding portions of the apex, with or without compression across the thickness of the apex.

### First LV Connector Construction

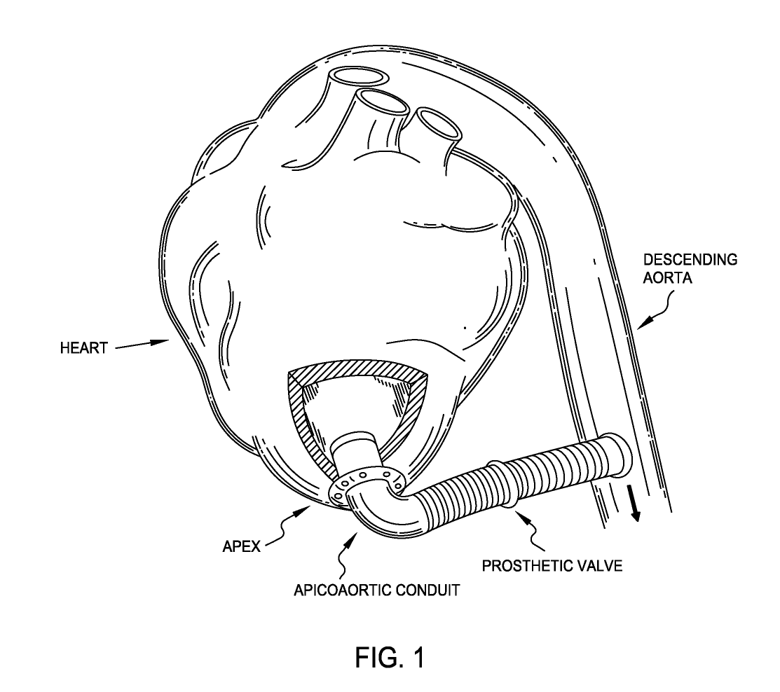

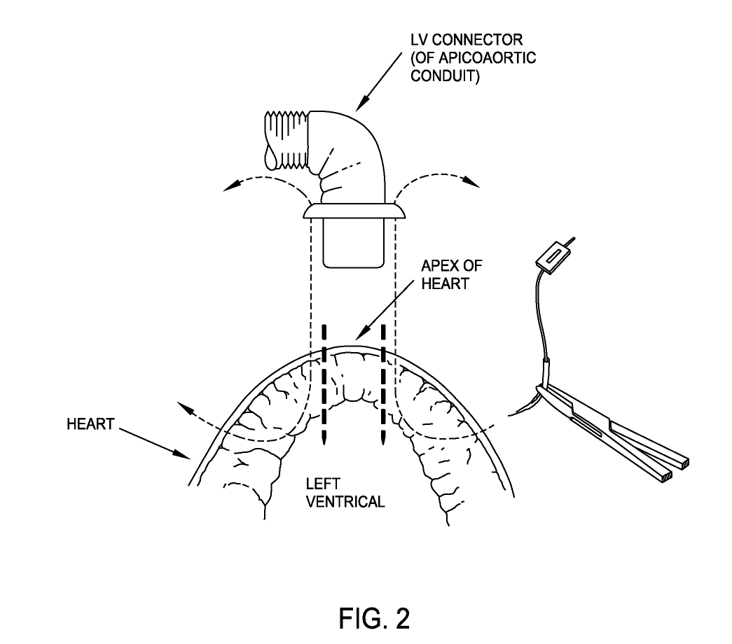

Referring again to FIG. 1, there is shown the general concept of an apicoaortic conduit, which extends from the apex of the left ventricle to the descending aorta, with a prosthetic valve positioned within the conduit.

In accordance with the present invention, the apicoaortic conduit comprises a left ventricle (LV) connector for connection to the apex of the heart, and a descending aorta connector (which contains the prosthetic valve) for connection to the descending aorta, with the LV connector being joinable to the descending aorta connector so as to form the complete apicoaortic conduit.

The present invention is intended to provide a novel LV connector capable of sutureless implantation in the apical wall of the heart.

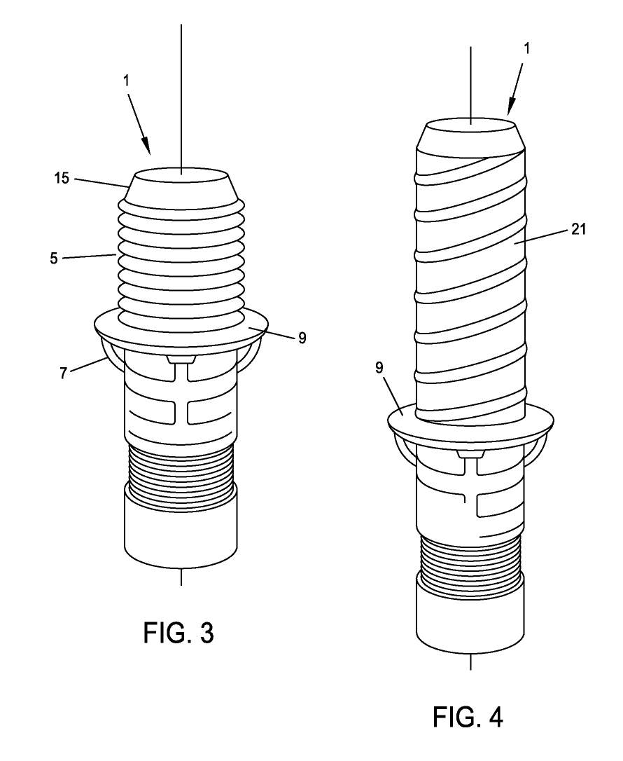

Looking next at FIGS. 3-9, there is shown a left ventricle (LV) connector 1 formed in accordance with the present invention. LV connector 1 comprises a hollow expandable stent comprising an internal spring 24. In this form of the invention, internal spring 24 preferably comprises a cylindrical coiled spring, i.e., a cylindrical tension spring (shown in FIG. 3 in an axially-contracted condition and in FIG. 4 in an axially-extended position). Cylindrical tension spring 24 is covered by a blood-retaining membrane 21 (e.g., fabric), stitched in place when the internal spring is in its axially-expanded condition, which assumes the shape of a series of folds 5 when internal spring 24 is in its axially-contracted condition (FIG. 3). The blood-retaining membrane 21 is straightened by the axial extension of internal spring 24. Behind the flange 9 are the securing lugs 7 which serve as part of the mechanism used to hold the internal spring in its axially-expanded (i.e., stretched) condition, as will hereinafter be discussed.

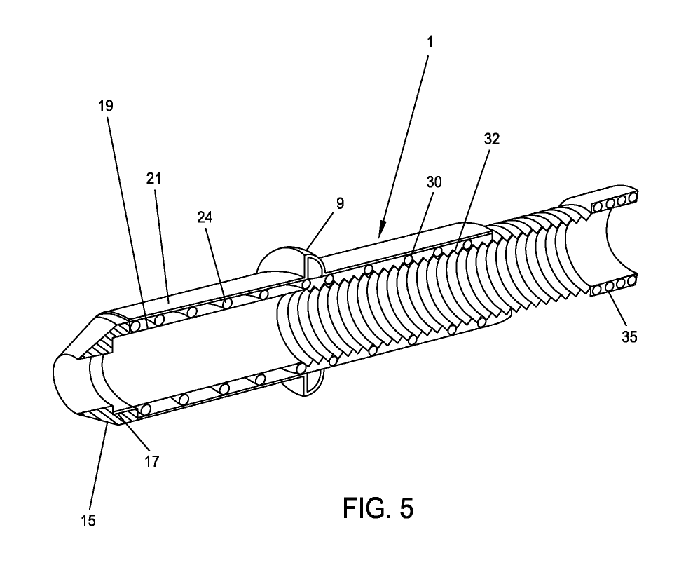

FIG. 5 is a perspective sectional view taken along the central axis of LV connector 1. Shoulder 17 on the inner surface of the tapered leading edge nosepiece 15 forms a part of the axially-expanding means for internal spring 24, as will hereinafter be discussed. Distal to flange 9, the internal spring is shown in its axially-expanded position; proximal to flange 9, the internal spring has a larger pitch 30 and is fully contracted in a more open condition. Distal to the flange, both inner membrane 19 and outer membrane 21 are shown in the axially-expanded condition. Behind flange 9, inner membrane 19 is in its normal pleated condition 32 and outer membrane 21 is in its normal non-folded condition 21. A coupling 35, for connecting to a corresponding coupling (not shown) on a descending aorta connector (also not shown) which is in fluid communication with the descending aorta, is shown at the end of LV connector 1 opposite nosepiece 15.

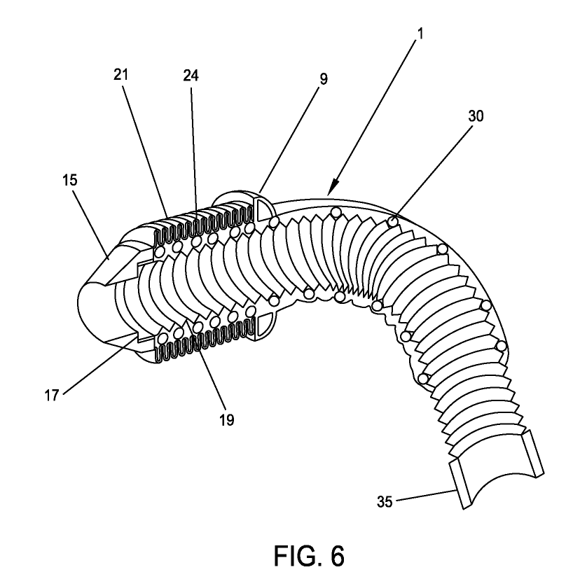

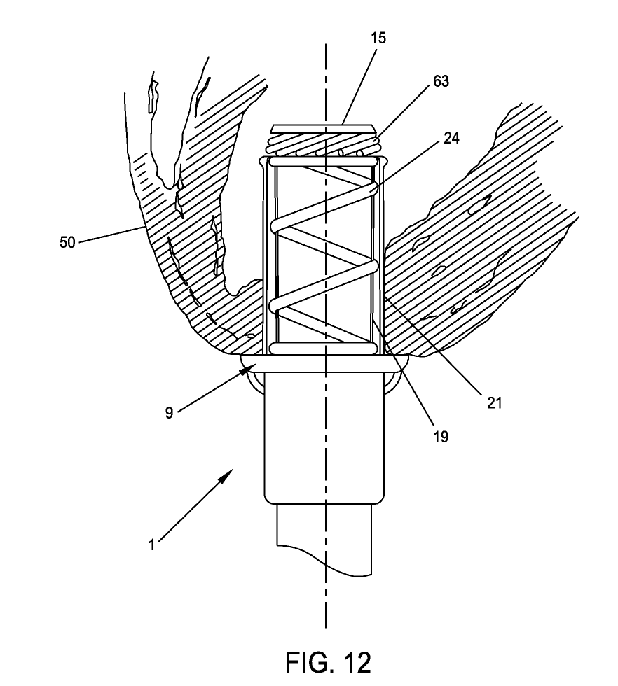

FIG. 6 shows LV connector 1 after internal spring 24 has been allowed to return to its axially-contracted condition. Outer membrane 21 responds to the contracting internal spring by forming a series of folds which significantly increase the radial diameter of the distal portion of the LV connector, e.g., by 15-20%. Inner membrane 19 also responds to the contracting internal spring by returning to its normal pleated configuration. The more open the pitch of the tension spring behind the flange, the more easily that the LV connector can be bent into a gentle curve. This curvature may be maintained by means of a drawstring (not shown) laced through the internal spring and tied off when the proper curve has been achieved. A similar arrangement for maintaining curvature is shown in FIGS. 12A and 12B of the aforementioned U.S. patent application Ser. No. 11/086,577.

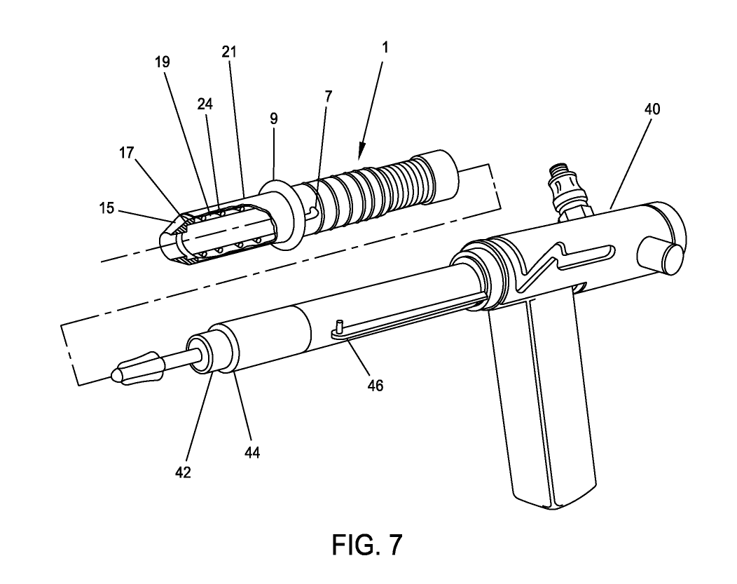

FIG. 7 shows, in partial section, LV connector 1 in its aforementioned axially-expanded condition (FIG. 5), with internal spring 24 extended and with inner membrane 19 and outer membrane 21 in their extended conditions. FIG. 7 also shows an applicator 40 which may be used to simultaneously form an opening in the side wall of the heart and set LV connector 1 into that opening. Applicator 40 is preferably of the sort disclosed in the aforementioned U.S. patent application Ser. Nos. 11/086,577, 11/581,081, 11/783,287 and 12/238,406. Applicator 40 is preferably modified so as to include a shoulder 44 on the distal end of the applicator to engage shoulder 17 of LV connector 1. Also included on applicator 40 is a latching mechanism 46 which engages lug 7 of the LV connector so that latching mechanism 46 can stretch LV connector 1 into its axially-expanded condition and hold it there until such time as it is to release the LV connector from that extended condition. Cutter 42 of applicator 40 extends beyond the nosepiece of the LV connector when the LV connector is properly mounted on applicator 40.

In use, the surgeon pushes and rotates applicator 40 so as to cause cutter 42 to core a hole into the left ventricle near the apex of the heart while simultaneously implanting LV connector 1 into that hole. Latching mechanism 46 is then released so that the internal spring 24 axially contracts, thereby forcing outer membrane 21 to fold up axially and thereby expand radially, thus locking the LV connector into the hole cored in the wall of the heart. Preferably internal spring 24 also increases radially as it contracts, thereby further binding the LV connector in the hole formed in the apical wall.

If desired, latching mechanism 46 of applicator (FIG. 7) could be configured so as to operate in a rotational manner about the longitudinal axis of applicator 40, as described in the aforementioned U.S. patent application Ser. Nos. 11/086,577, 11/581,081, 11/783,287 and 12/238,406.

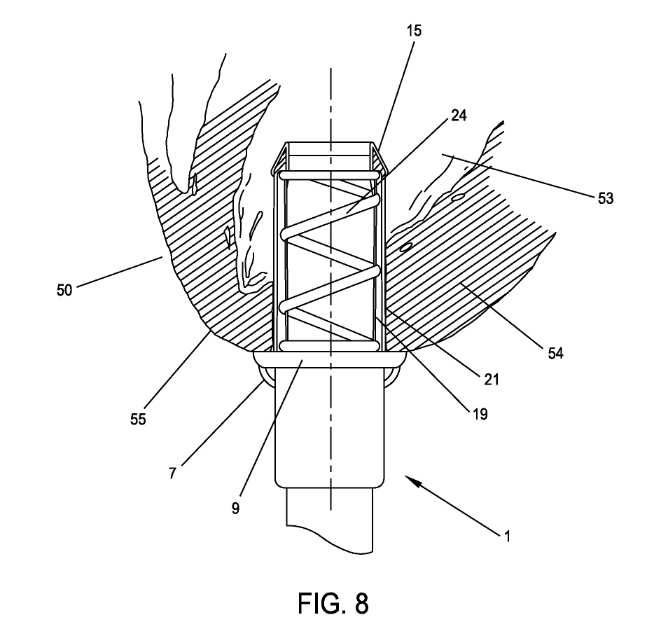

FIG. 8 is a sectional view of the lower portion of the left ventricle 53 of the heart 50, with LV connector 1 inserted in a hole cut in the heart. With internal spring 24 still in its axially-expanded (i.e., stretched) condition, inner membrane 19 and outer membrane 21 are also in their axially-expanded conditions. The LV connector has been inserted into the heart wall 54 slightly off-axis from the apex of the heart 55. The flange 9 is flush with the outside of the heart. Applicator 40 (with its cutter 42) has been removed from the LV connector in FIG. 8 for clarity of viewing. Since cutter 42 resides within the interior of the LV connector during formation of the apical hole and insertion of the LV connector into that formed hole, cutter 42 is necessarily smaller than the outside diameter of LV connector 1. Thus, the LV connector is always implanted in a hole which has a smaller diameter than that of the LV connector itself, thereby always resulting in an interference or press fit with the tissue of the heart.

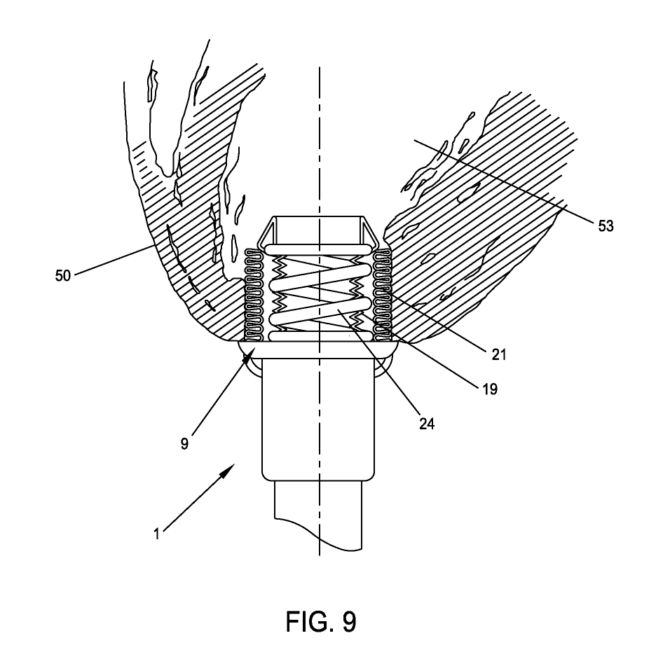

FIG. 9 is a view similar to that of FIG. 8, except that the internal spring 24 has been released. As a result, the contracting spring forces outer membrane 21 to fold-up axially and expand radially, thereby further locking the LV connector into the hole cored in the heart. At the same time, inner membrane 19 returns to its original pleated configuration. Preferably internal spring 24 increases radially as it contracts, thereby further binding LV connector 1 in the formed hole.

Thus it will be seen that, with this form of the invention, an interference fit is initially created between the LV connector and the side wall of the formed hole by virtue of the fact that cutter 42 of applicator 40 has a smaller diameter than the LV connector. This interference fit is then significantly supplemented by the radial expansion of outer membrane 21 when internal spring 24 axially contracts. This binding fit is then further significantly supplemented by radial expansion of the spring itself as the internal spring axially contracts.

### Second LV Connector Construction

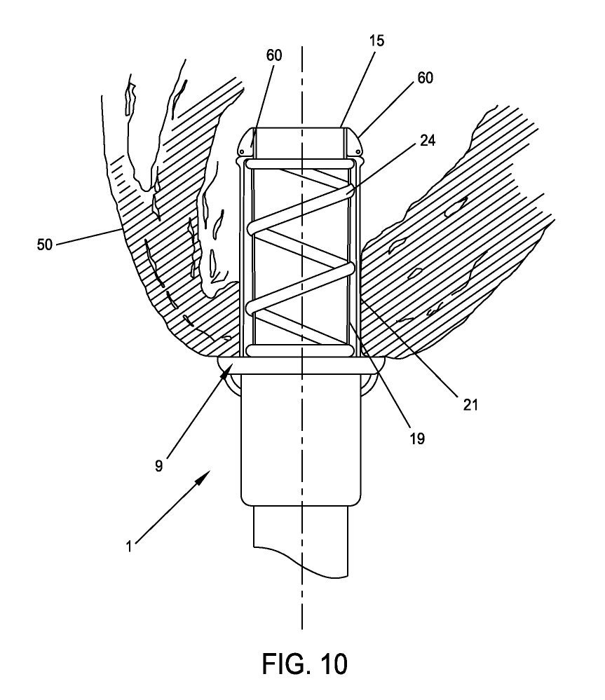

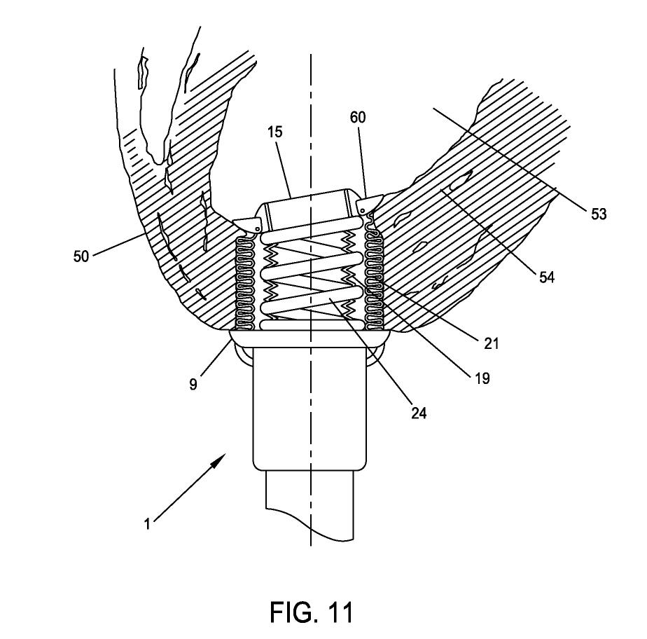

FIGS. 10 and 11 show another LV connector formed in accordance with the present invention. The LV connector shown in FIGS. 10 and 11 is generally similar to the LV connector shown in FIGS. 3-9, except that in this form of the invention, the tapered distal end 15 of the LV connector has been fitted with at least one radially-expandable element, preferably in the form of a plurality of pivotable arms 60.

FIG. 10 shows the LV connector with its radially-deployable pivotable arms 60 in their retracted position. With internal spring 24 in its axially-expanded (i.e., stretched) position, inner membrane 19 and outer membrane 21 are also in their axially-expanded position. Flange 27 is flush with the outside of the heart. Again, in FIG. 10, applicator 40 (with its cutter) has been removed from the LV connector for clarity of viewing.

FIG. 11 shows the same LV connector, but with internal spring 24 in its axially-contracted position, and with pivotable arms 60 deployed outwardly. As a result, the axially-contracting internal spring 24 forces outer membrane 21 to fold-up axially and expand radially, thereby locking the LV connector into the hole formed in the heart. This binding action is further supplemented by any radial expansion of internal spring 24 as the spring contracts axially. Inner membrane 19 returns to its original pleated configuration. Since pivotable arms 60 have been deployed, as internal spring 24 axially contracts, it pulls the larger diameter of the deployed pivotable arms 60 against the inner surface of the ventricle wall 54. Significantly, the flexibility of internal spring 24 allows the deployed pivotable arms to adjust to the uneven, unparallel inner surface of the heart, with each coil of the internal spring adjusting as needed so as to distribute any uneven forces. The larger diameter of pivotable arms 60 thus provides a counter-tension element to the flange 9 as internal spring 24 pulls pivotable arms 60 and flange 9 towards each other. The effect of these inner and outer counter-tension devices (i.e., pivotable arms 60 and flange 9) pulling together is to put pressure on the layers of the heart wall, thereby helping to prevent pseudoaneurysms. The large diameter of the deployed pivotable arms 60 also prevents implant pull-out while the radial outward pressure of folded membrane 21 (and the radially-expanded internal spring 24) aids in preventing pull-out and provides hemostasis.

### Third LV Connector Construction

FIGS. 12-14 show another embodiment of the present invention formed in accordance with the present invention. The LV connector shown in FIGS. 12-14 is generally similar to the LV connector shown in FIGS. 10 and 11, except that this form of the invention, the tapered distal end 15 of the LV connector has been fitted with a plurality of radially-deployable finger arms 63.

FIG. 12 shows the LV connector with its radially-deployable finger arms 63 in their retracted position wherein they are tightly wrapped around the tapered end of the LV connector. In FIG. 12, the LV connector is shown inserted into a hole formed in the heart, with internal spring 24 still in its axially-expanded (i.e., stretched) condition, so that inner membrane 19 and outer membrane 21 are also in their axially-expanded conditions. Flange 9 is flush with the outside of the heart. Again, in FIG. 12, applicator 40 (with its cutter) has been removed from the LV connector for clarity of viewing.

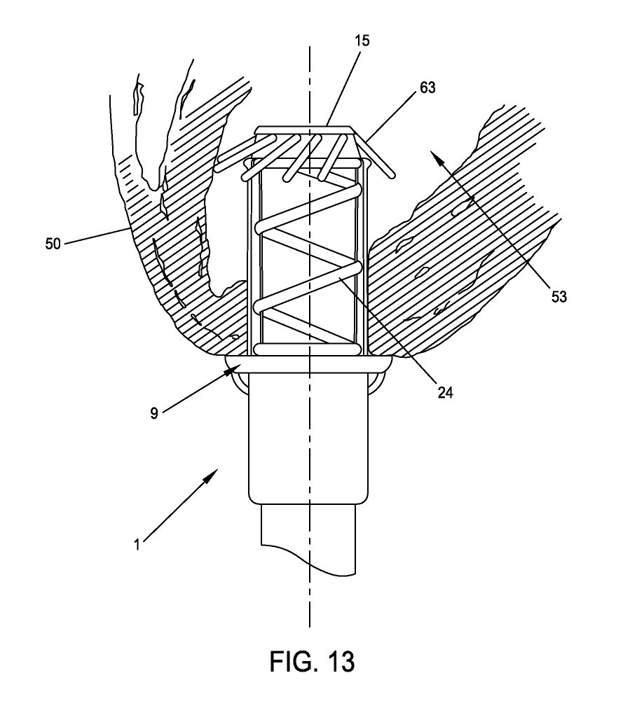

FIG. 13 shows radially-deployable finger arms 63 deployed outwardly before internal spring 24 is released. Radially-deployable finger arms 63 themselves act as small springs and self-deploy when released, e.g., by removing a mechanical constraint. As illustrated in FIG. 13, radially-deployable finger arms 63 spring radially outwardly and axially downwardly when released. Radially-deployable finger arms 63 may also coil outwardly, or they may be inflatable (e.g., with saline).

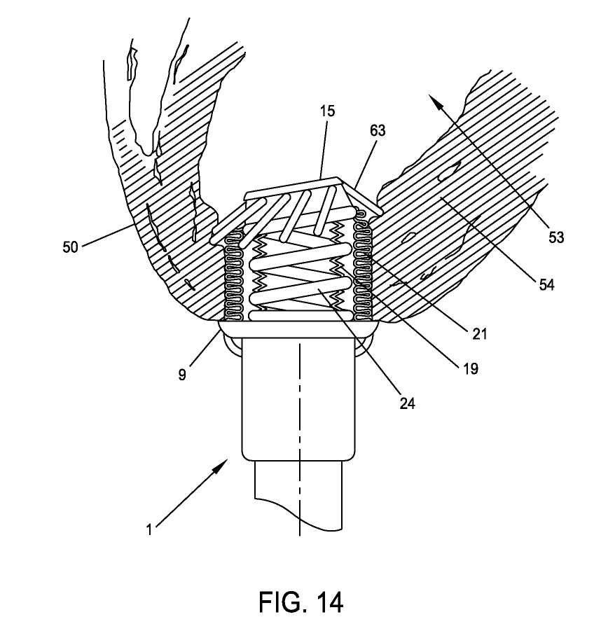

FIG. 14 shows the LV connector after internal spring 24 has been released. The contracting spring forces outer membrane 21 to fold up axially and expand radially, thereby locking the LV connector into the hole formed in the heart. This binding is further supplemented by any radial expansion of internal spring 24 as the spring contracts axially. Inner membrane 19 returns to its original pleated configuration. As internal spring 24 contracts, it pulls the larger diameter of the deployed finger arms 63 against the inner surface of the ventricle wall 54. Internal spring 24 allows the deployed finger arms to adjust to the uneven, unparallel inner surface of the heart, with each coil of the spring adjusting as necessary so as to distribute any uneven forces. The larger diameter of deployed finger arms 63 provides a counter-tension element to flange 9 as internal spring 24 pulls the elements towards each other. The effect of the inner and outer counter-tension devices (i.e., deployed spring arms 63 and flange 9) pulling together is to put pressure on the layers of the heart wall, thereby helping to prevent pseudoaneurysms. The large diameter of the radially-deployable pivot arms 63 prevents implant pull-out while the radial outward pressure of the folded membrane 21 (and the radially-expanded internal spring 24) aids in preventing pull-out and provides hemostasis.

### Fourth LV Connector Construction

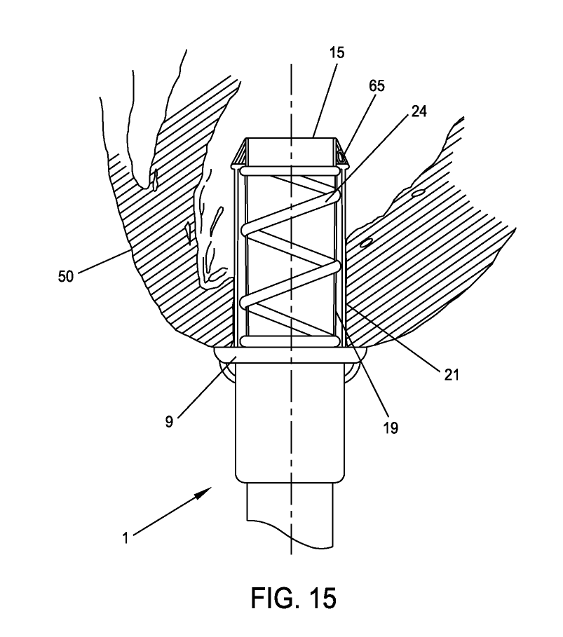

FIGS. 15-17 show another LV connector formed in accordance with the present invention. The LV connector shown in FIGS. 15-17 is generally similar to that the LV connectors shown in FIGS. 10 and 11 and FIGS. 12-14, except that in this form of the invention, the tapered distal end 15 of the LV connector is fitted with a radially-deployable balloon element 65.

FIG. 15 shows the LV connector with its radially-deployable balloon element 65 in its retracted and un-inflated position, tightly wrapped around the tapered end of the LV connector. In FIG. 15, the LV connector is shown inserted in a hole formed in the heart. With internal spring 24 in its axially-expanded (i.e., stretched) condition, inner membrane 19 and outer membrane 21 are also in their axially-expanded positions. Flange 9 is flush with the outside of the heart. In FIG. 15, applicator 40 (with its cutter) has been removed from the LV connector for clarity of viewing.

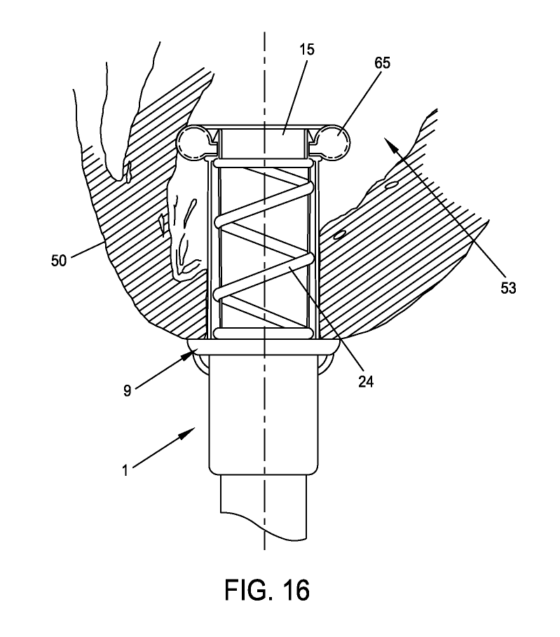

FIG. 16 shows the LV connector of FIG. 15 after radially-deployable balloon element 65 has been deployed but before the internal spring has been released. The radially-deployable balloon element could be inflated with, for example, saline. The radially-deployable balloon element could also be inflated with an implantable epoxy material that would “set up” after deployment. Radially-deployable balloon element 65 may be inflated by, for example, a syringe or other pump device suitable for the chosen inflation medium.

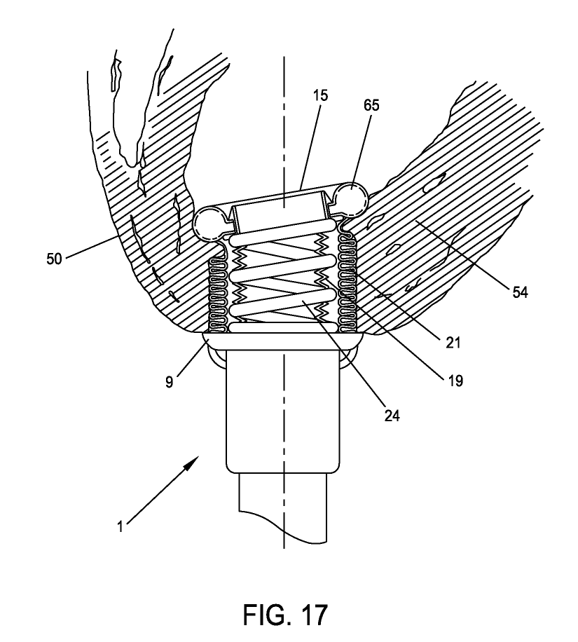

FIG. 17 shows the LV connector of FIG. 15 after internal spring 24 has been released. The contracting spring forces outer membrane 21 to fold up axially and to expand radially, thereby locking the LV connector into the hole formed in the heart. This binding is further supplemented by any radial expansion of internal spring 24 as the spring contracts axially. Inner membrane 19 returns to its original pleated configuration. As internal spring 24 contracts, it pulls the larger diameter of the deployed balloon element 65 against the inner surface of the ventricle wall 54. Internal spring 24 allows the deployed balloon element 65 to adjust to the uneven, unparallel inner surface of the heart, with each coil of the spring adjusting as necessary so as to distribute any uneven forces. The larger diameter of deployed balloon 65 provides a counter-tension element to flange 9 as internal spring 24 pulls them towards each other. The effect of these inner and outer counter-tension devices (i.e., inflated balloon element 65 and flange 9) pulling together is to put pressure on the layers of the heart wall, thereby helping to prevent pseudoaneurysms. The large diameter of deployed balloon element 65 also prevents implant pull-out while the radial outward pressures of folded membrane 21 (and the radially-expanded internal spring 24) aids in preventing pull-out and provides hemostasis.

### Fifth LV Connector Construction

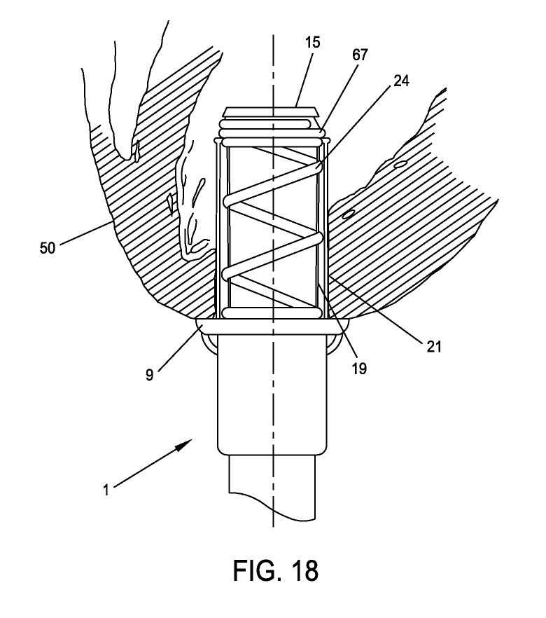

FIGS. 18-20 show another LV connector formed in accordance with the present invention. The LV connector shown in FIGS. 18-20 is generally similar to the LV connector shown in FIGS. 10 and 11, 12-14 and 15-17, except that in this form of the invention, the tapered distal end 15 of the LV connector has been fitted with a deployable conical helical spring element 67.

FIG. 18 shows the LV connector with its deployable conical helical spring element 67 in its retracted position, tightly wrapped around the tapered end of the LV connector and held in position by a release wire (not shown). In FIG. 18, the LV connector is shown implanted in a hole formed in the heart, with internal spring 24 in its axially-expanded (i.e., stretched) condition, so that inner membrane 19 and outer membrane 21 are also in their axially-expanded conditions. Flange 9 is flush with the outside of the heart. In FIG. 18, applicator 40 (with its cutter) has been removed from the LV connector for clarity of viewing.

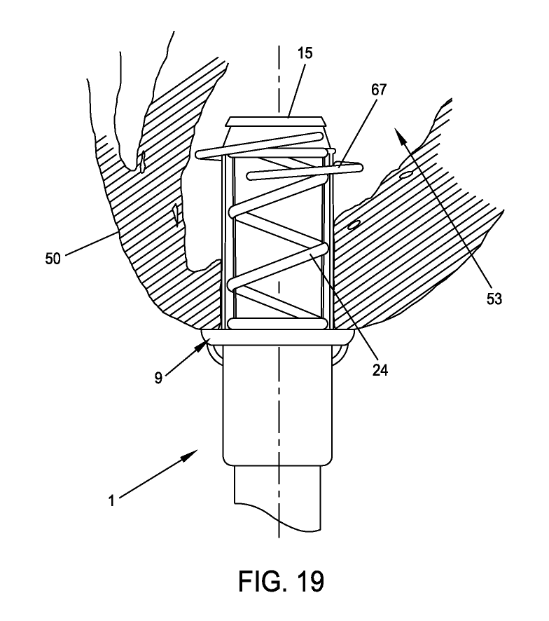

FIG. 19 shows the LV connector of FIG. 18 after conical helical spring element 67 has been released and deployed, but before internal spring 24 is released. As illustrated, conical helical spring element 67 self-deploys when released, springing radially outwardly and axially downwardly. For containment purposes, conical helical spring element 67 may be enclosed in a fitted jacket of graft material.

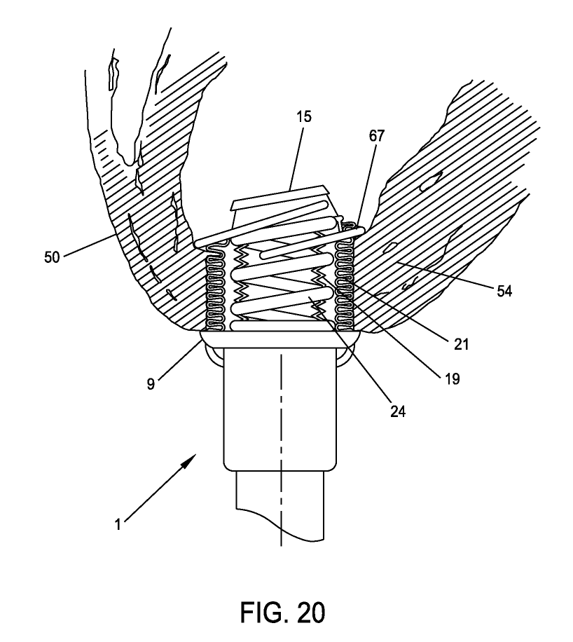

FIG. 20 shows the implanted LV connector of FIG. 19 after internal spring 24 has been released. The contracting spring forces outer membrane 21 to fold up axially and expand radially, thereby locking the LV connector into the hole formed in the heart. This binding is further supplemented by any radial expansion of internal spring 24 as the spring contracts axially. Inner membrane 19 returns to its original pleated configuration. As internal spring 24 contracts, it pulls the larger diameter of the deployed conical helical spring element 67 against the inner surface of the ventricle wall 54. Internal spring 24 allows conical helical spring element 67 to adjust to the uneven, unparallel inner surface of the heart, with each coil of the internal spring adjusting as necessary so as to distribute any uneven forces. The larger diameter of deployed conical helical spring element 67 provides a counter-tension element to flange 9 as internal spring 24 pulls the elements towards each other. The effect of the inner and outer counter-tension devices (i.e., conical helical spring element 67 and flange 9) pulling together is to put pressure on the layers of the heart wall, thereby helping to prevent pseudoaneurysms. The large diameter of conical helical spring element 67 prevents implant pull-out while the radial outward pressures of folded membrane 21 (and the radially expanded internal spring 24) aids in preventing pull-out and provides hemostasis.

### Sixth LV Connector Construction

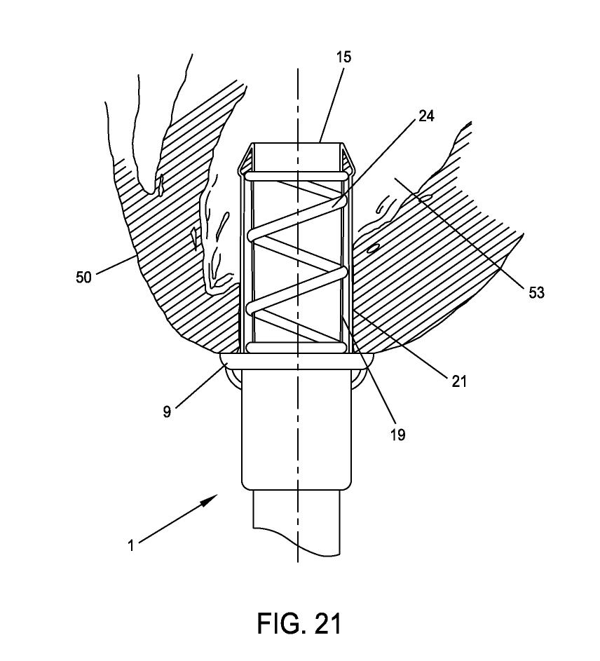

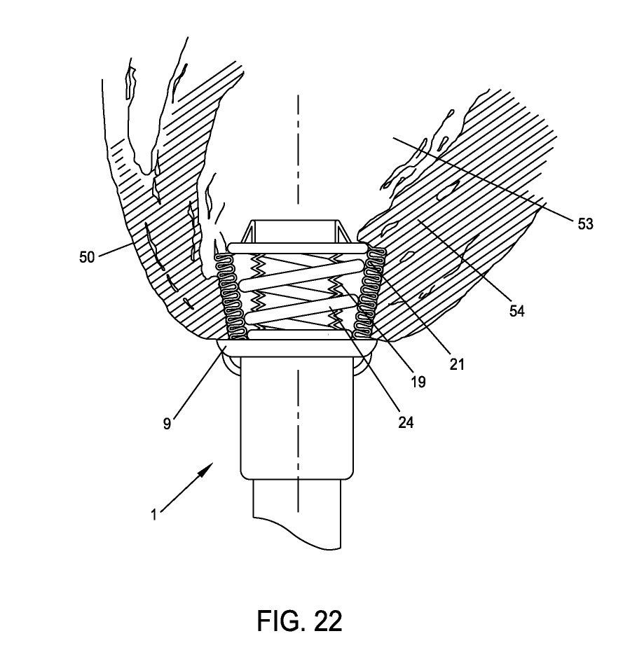

FIGS. 21 and 22 show another LV connector formed in accordance with the present invention. The LV connector shown in FIGS. 21 and 22 uses a somewhat different construction from that of FIGS. 3-9, 10 and 11, 12-14, 15-17 and 18-20. More particularly, in this form of the invention, the LV connector comprises a hollow expandable stent comprising a frusto-conical spring 24 which is capable of being expanded axially and torsioned radially, so that it expands radially as it contracts longitudinally.

More particularly, in FIG. 21, the LV connector is shown implanted in a hole formed in the heart. With frusto-conical spring 24 still in its axially-expanded (and radially-torsioned) condition, inner membrane 19 and outer membrane 21 are also in their axially-expanded conditions. Flange 9 is flush with the outside of the heart. In FIG. 21, applicator (with its cutter) has been removed from the LV connector. As noted above, in this form of the invention, frusto-conical spring 24 is capable of being both expanded axially and torsioned radially. In its axially-expanded and radially-torsional condition, the frusto-conical spring has a generally cylindrical configuration, (FIG. 21), whereas in its relaxed condition (FIG. 22), the frusto-conical spring has an inverted frusto-conical configuration, with the larger diameter inside the left ventricle 53 of the heart. The internal spring may be held in its generally cylindrical configuration and released by a cable release mechanism which pins the free end of the internal spring.

FIG. 22 shows the LV connector of FIG. 21 when frusto-conical spring 24 is released from its axially-expanded (and radially-torsional) condition. The frusto-conical spring “self-deploys” in the sense that, upon release, it radially unwinds into its frusto-conical configuration while simultaneously contracting in the axial direction. Outer membrane 21 contracts into a series of folds while the inner membrane 19 returns to its original pleated configuration.

In this embodiment, and as shown in FIG. 22, the distal end of frusto-conical spring 24 is itself expanding significantly in the radial direction, thereby creating an inverted cone shape that presses against the side wall of the hole formed in the heart and creating a resultant force that is directed into left ventricle 53. The inward force created by the conical helical internal spring 24 is countered by the flange 9 on the outside of the heart.

### Seventh LV Connector Construction

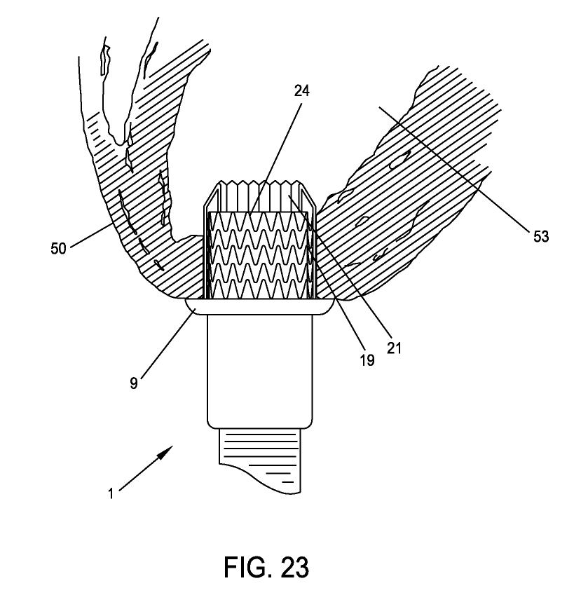

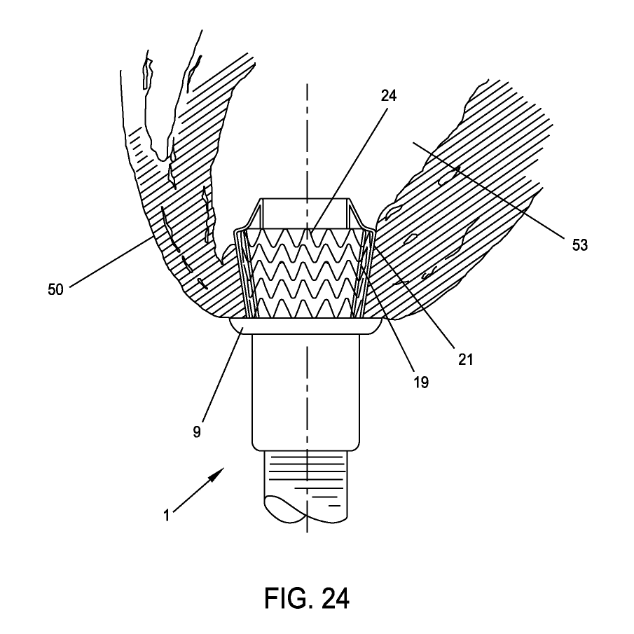

FIGS. 23 and 24 show another LV connector formed in accordance with the present invention. The LV connector shown in FIGS. 23 and 24 comprises a hollow expandable stent in the form of a frusto-conical structure 24, wherein the frusto-conical structure comprises a Z-stent. More particularly, the Z-stent preferably comprises Nitinol wire having a zigzag (or “Z-wire”) configuration and which has been treated on a mandrel so as to form a substantially frusto-conical structure when the Z-stent is in its unconstrained condition.

As seen in FIG. 23, frusto-conical structure 24 is compressed radially into a cylindrical shape of the desired diameter, and implanted into the hole formed in the left ventricle 53 until flange 9 is flush with the outside of the heart. In FIG. 23, applicator 40 (with its cutter) has been removed from the LV connector for clarity of viewing. Inner membrane 19 and outer membrane 21 are shown in a vertically pleated or folded condition. This pleating allows for radial expansion of the membranes when a releasing mechanism allows the compressed frusto-conical structure to resume its normal (i.e. relaxed, conically-shaped) condition.

FIG. 24 shows the LV connector of FIG. 23 when frusto-conical structure 24 is released from its compressed condition. The frusto-conical structure self-deploys in the sense that, upon release, the frusto-conical structure radially expands into its preferred conical configuration. Inner membrane 19 and outer membrane 21 are stretched radially out from their vertically pleated configuration until they are pressed tightly against the side wall of the hole formed in the heart.

In this form of the invention, and as shown in FIG. 24, the distal end of frusto-conical structure 24 is itself expanding significantly in the radial direction, thereby creating an inverted cone shape that presses against the hole formed in the heart and creating a resultant force that is directed into the left ventricle 53. The inward force created by conical frusto-conical structure 24 is countered by flange 9 on the outside of the heart.

### Eighth LV Connector Construction

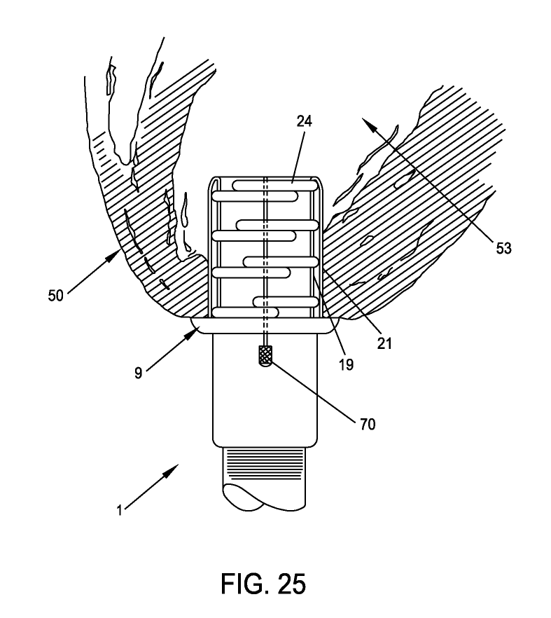

FIGS. 25-27 show another LV connector formed in accordance with the present invention. The LV connector shown in FIGS. 25-27 comprises a hollow expandable stent in the form of a frusto-conical structure 24, wherein the frusto-conical structure comprises a series of torsional springs which together form a generally conical tubular shape.

As seen in FIG. 25, these torsional springs can be torsionally contracted into a generally cylindrical configuration, and then held in place by a locking pin 70. Alternatively, if desired, locking pin 70 may also hold the frusto-conical structure in a compressively extended position.

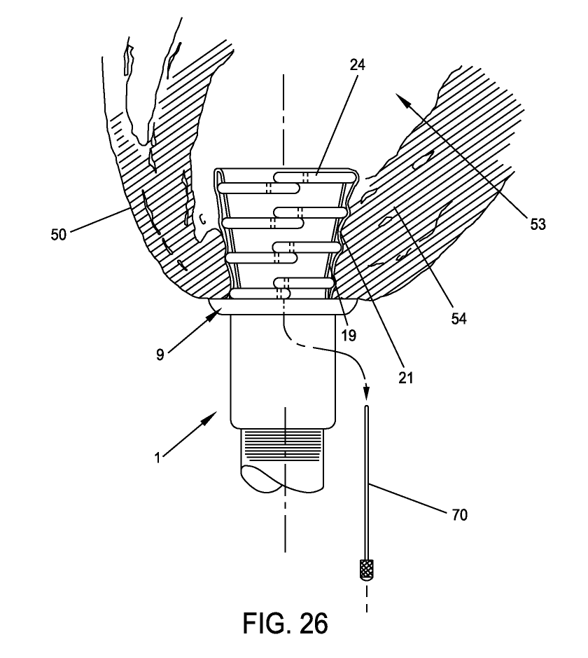

FIG. 26 shows the LV connector of FIG. 25 when frusto-conical structure 24 is released by disengaging retaining pin 70. The frusto-conical structure self-deploys, in the sense that upon release, it radially expands into its preferred conical configuration. Outer membrane 21 preferably “contracts” about the expanding torsional springs so as to form a substantially ribbed frusto-conical structure. This ribbing helps to tightly bind the LV connector into the hole formed in the wall of the heart.

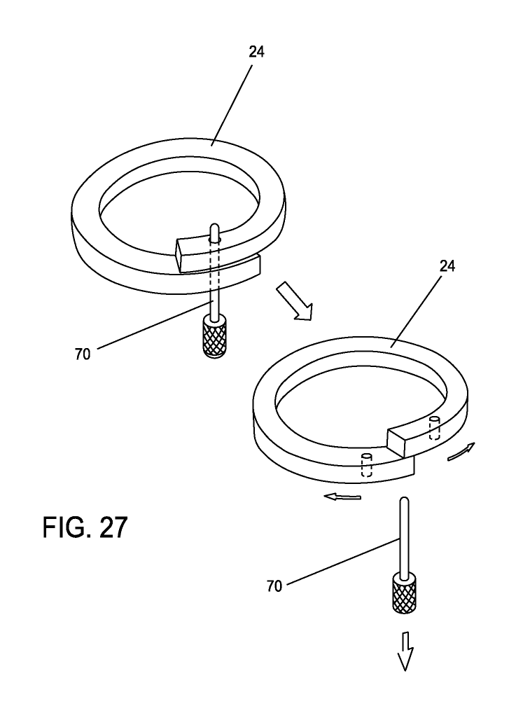

FIG. 27 shows the action of a single torsional spring element. In the upper illustration, the torsional spring element is held in coiled torsional tension by retaining pin 70. In the lower illustration, retaining pin 70 has been removed from the torsional spring element. Removing the retaining action of pin 70 causes the torsional spring element to unwind to its pre-tensioned configuration, thereby increasing its diameter.

### Ninth LV Connector Construction

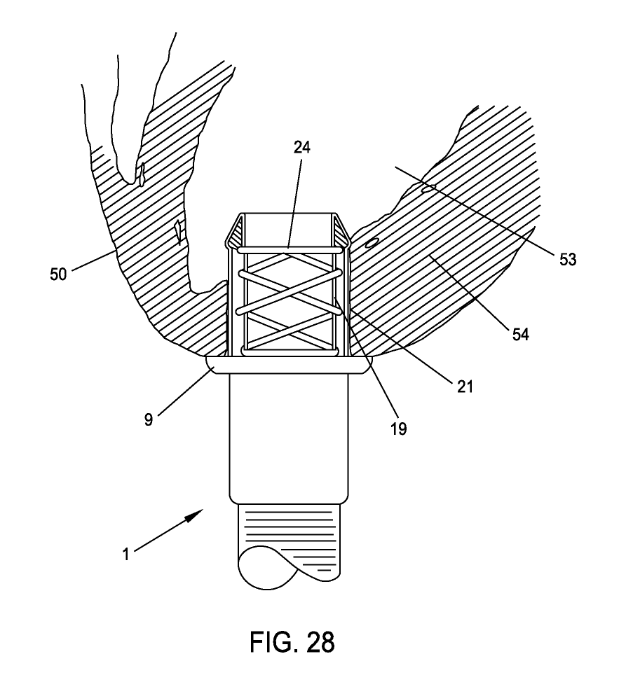

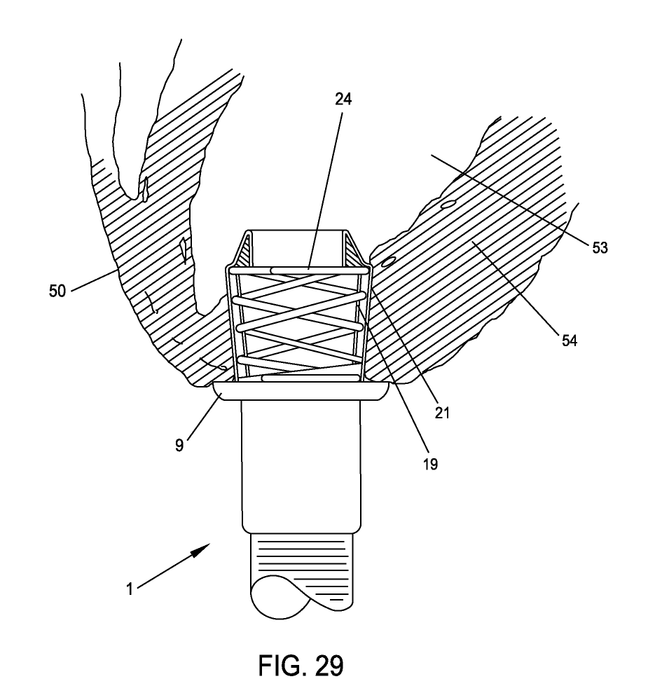

FIGS. 28 and 29, show another LV connector formed in accordance with the present invention. The LV connector shown in FIGS. 28 and 29 is generally similar to the LV connectors shown in FIGS. 21 and 22, except that in this form of the invention, the hollow expandable stent comprises a frusto-conical structure 24 which consists of two or more frusto-conical torsion springs wound in opposite directions.

As seen in FIG. 28, the two or more torsional springs may be forced into a torsionally contracted condition, and then held in place by a locking pin, e.g., a locking pin similar to the locking pin 70 of FIG. 25. Alternatively, the locking pin might be contained within cutter 42 and extend through the cutter so as to secure the multiple torsion springs in place on the cutter.

FIG. 29 shows that the multiple, oppositely-directed torsion springs, when released, self-deploy by spiraling outward so as to assume their normal conical helical configuration, thereby applying force against the hole formed in the heart and creating a resultant force that is directed into the left ventricle 53. The inward force created by the frusto-conical structure 24 is countered by the flange 9 on the outside of the heart. This action tightly binds the LV connector into the hole formed in the wall of the heart.

### Tenth LV Connector Construction

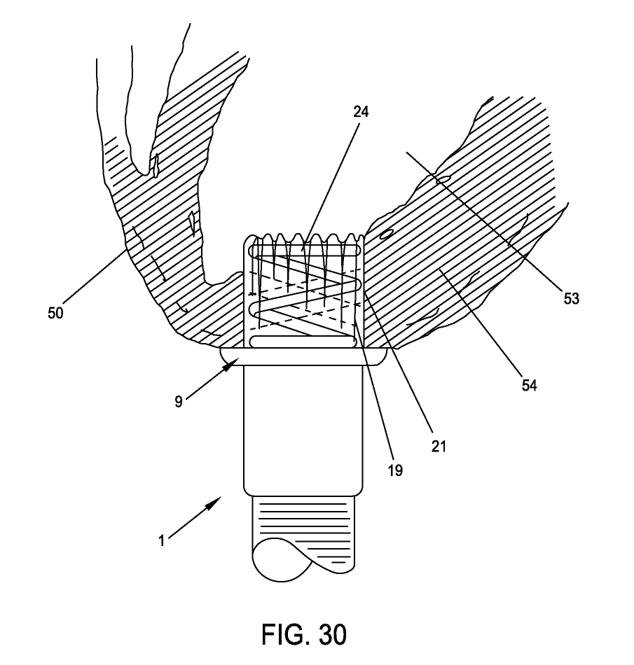

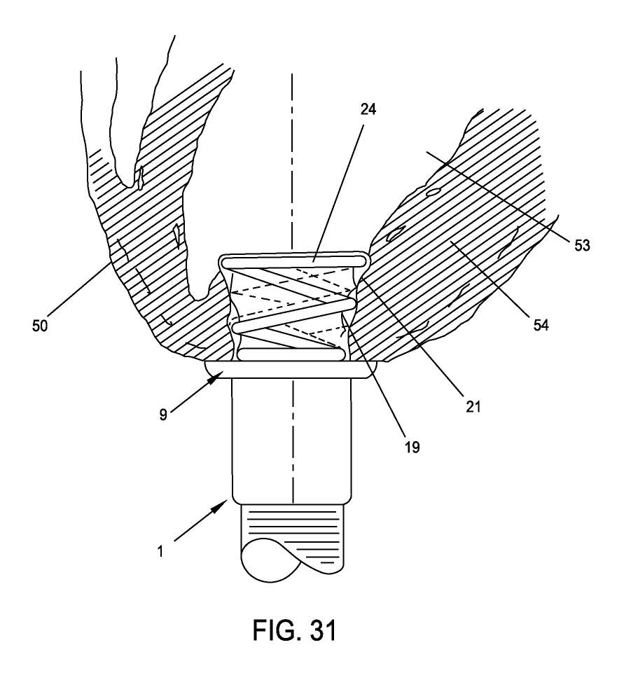

FIGS. 30 and 31 show another LV connector formed in accordance with the present invention. The LV connector shown in FIGS. 30 and 31 comprises a hollow expandable stent comprising a frusto-conical spring 24. In this form of the invention, frusto-conical spring 24 preferably comprises a torsional spring.

As seen in FIG. 30, the torsional spring may be forced into a torsionally-contracted (i.e., cylindrical) condition, and then held in place by a locking pin, e.g., a locking pin similar to the locking pin 70 of FIG. 25. Inner membrane 19 and outer membrane 21 are shown in FIG. 28 in a vertically pleated or folded condition. This pleating allows for radial expansion of inner membrane 19 and outer membrane 21 when the retaining pin (or other releasing mechanism) allows the internal spring to resume its normal (i.e., unbiased) condition.

FIG. 31 shows the LV connector of FIG. 30 when frusto-conical spring 24 is released from its constrained condition, e.g., by disengaging a locking pin similar to the locking pin 70 of FIG. 25. Frusto-conical spring 24 self-deploys, in the sense that, upon release, it radially unwinds into its preferred conical helical configuration. Outer membrane 21 and inner membrane 19 expand radially outwardly, losing their initial pleated configuration and accommodating the formation of shallow indents in the membrane between the coils of the internal spring. This indented membrane surface provides a “ribbed effect” which allows for better engagement with the tissue surface, while the expanded conical shape of frusto-conical spring 24, and the expanded conical membranes 19 and 21, serve to prevent the LV connector from coming out of the hole formed in the heart.

### Eleventh LV Connector Construction

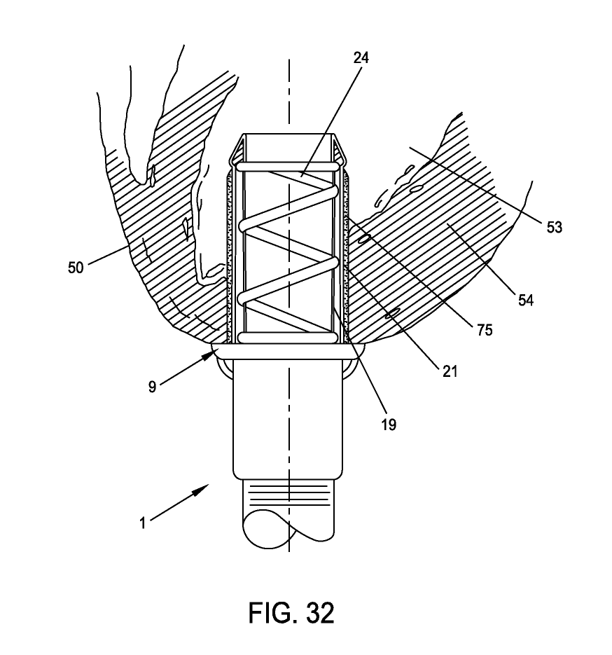

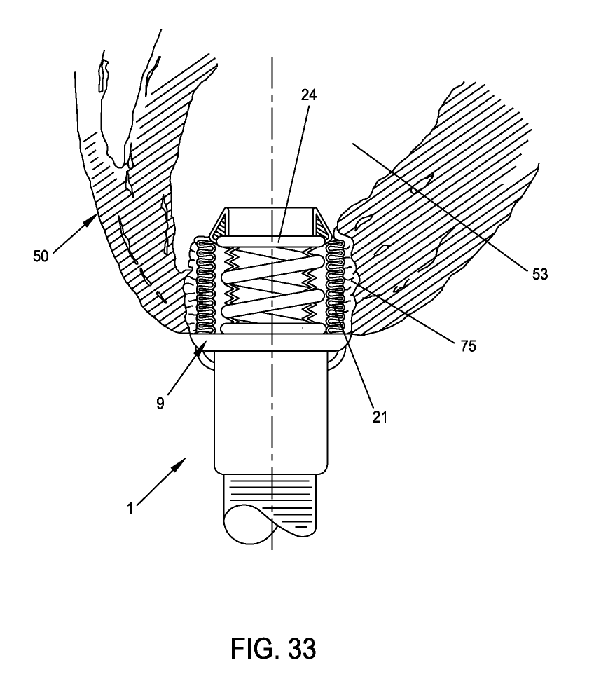

FIGS. 32 and 33 show another LV connector formed in accordance with the present invention. The LV connector shown in FIGS. 32 and 33 is generally similar to the LV connector shown in FIGS. 3-9, except that in this form of the invention, outer membrane 21 is surrounded by a layer of hydrophilic foam 75, or a material made, for example, of isocyanate-capped polyester pre-polymer, or various compositions of a polyelectrolyte and polyvinyl alcohol, or various implantable hydrogels, especially bioresorbable hydrogels or keratin-type hydrogels used for tissue expansion.

FIG. 32 shows the LV connector when its cylindrical coiled spring 24 is in its axially-expanded (i.e., stretched) condition.

FIG. 33 shows the LV connector when its cylindrical spring 24 is released. Outer membrane 21 responds to the contracting cylindrical spring 24 by forming a series of folds which increase the radial diameter of the distal portion of the LV connector and consequently increase the radial force applied to the hole formed in the heart. The outer layer of hydrophilic material 75 likewise contracts into a series of folds, further increasing the radial outward pressure on the side wall of the hole formed in the heart.

Significantly, the outer hydrophilic layer of material begins to absorb water from the blood in the left ventricle 53 and expands, further holding the LV connector implant tightly in the formed hole.

In addition, if the hydrophilic material is impregnated with, for example, collagen fibers, then the expanding hydrophilic layer 75 will also expedite the clotting cascade, thereby sealing any blood pathways which might exist around the tightly implanted LV connector.

Thus, the hydrophilic foam layer 75 not only acts to prevent the LV connector from coming out of the formed hole, but also acts to speed the process of clotting and, ultimately, tissue in-growth.

It should be appreciated that the foam, shown as a uniform layer in FIG. 32, could be made in a variety of shapes and, in particular, could be made thinner near the outer heart wall and thicker near the inner heart wall. The foam layer could also be compressed in such manner that, in a dry state, it would appear to be of uniform thickness but which would, thereafter, assume a wedge-like form as water is absorbed by the hydrophilic layer, thereby locking the LV connector even more securely in the heart wall.

It should also be appreciated that, although the hydrophilic layer is shown here (for clarity) with a particular embodiment of LV connector, the outer hydrophilic layer will work similarly with any LV connector construction herein disclosed.

### Twelfth LV Connector Construction

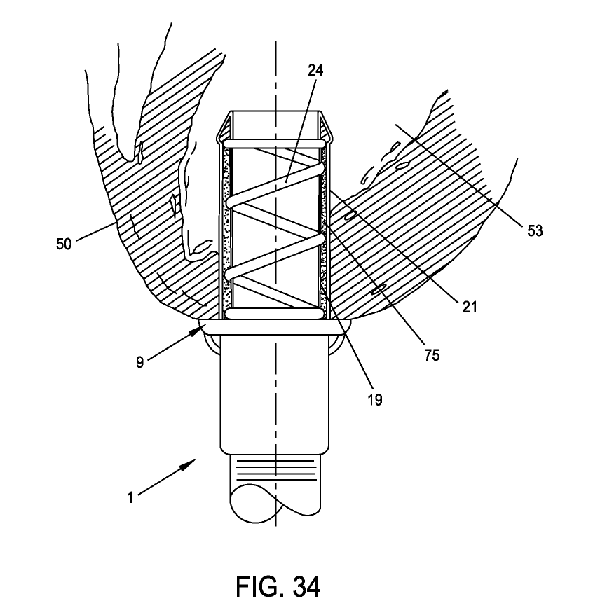

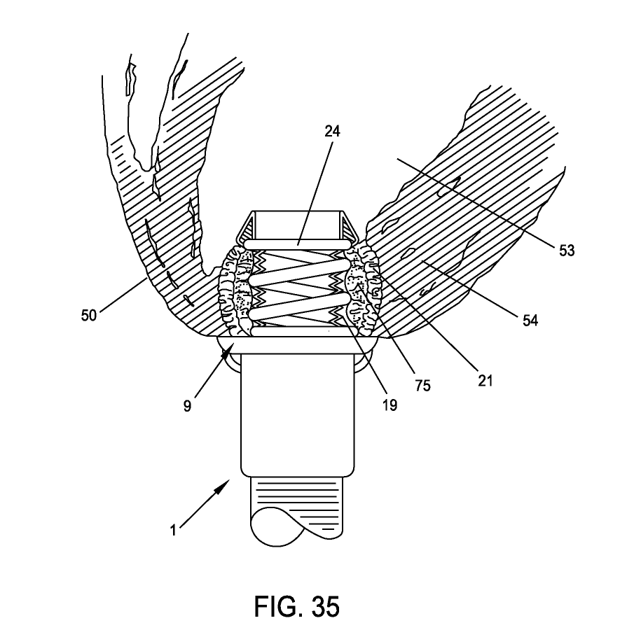

FIGS. 34 and 35 show another LV connector formed in accordance with the present invention. The LV connector shown in FIGS. 34 and 35 is generally similar to the LV connector shown in FIGS. 3-9, except that in this form of the invention, a layer of hydrophilic foam or material is disposed between outer membrane 21 and inner membrane 19. The hydrophilic foam or material may be made, for example, of isocyanate-capped polyester pre-polymer or various compositions of a polyelectrolyte and polyvinyl alcohol, or various implantable hydrogels, especially bioresorbable hydrogels or keratin-type hydrogels used for tissue expansion.

FIG. 34 shows the LV connector when its cylindrical coiled spring is in its axially-expanded (i.e., stretched) condition.

FIG. 35 shows the LV connector when cylindrical coiled spring 24 is released. Outer membrane 21 responds to the contracting cylindrical coiled spring by forming a series of folds which increase the radial diameter of the distal portion of the LV connector and, consequently, increase the radial force applied to the hole formed in the heart. The middle layer of hydrophilic material 75 likewise contracts into a series of folds, further increasing the radial outward pressure on the side wall of the hole formed in the heart. Then the middle hydrophilic layer of material begins to absorb water from the blood in the left ventricle 53 and the cut heart wall 54 and expands, much in the manner of a boat plug, thereby further holding the LV connector implant tightly in the hole formed in the heart. The contracted coils of cylindrical coiled spring 24, and the inner membrane 19, prevent the expanding hydrophilic layer from partially occluding the lumen of the LV connector. An additional layer of membrane (not shown) can be provided between the hydrophilic layer and the internal spring to further prevent intrusion by the hydrophilic material into the lumen of the LV connector.

Furthermore, if the hydrophilic material is impregnated with, for example, collagen fibers, then the expanding hydrophilic layer 75 will also expedite the clotting cascade, thereby sealing any blood pathways that might exist around the tightly implanted LV connector. Thus, the hydrophilic foam layer 75 not only acts to prevent the LV connector from coming out of the hole formed in the heart, but also acts to speed the process of clotting and, ultimately, tissue in-growth.

It should be appreciated that the foam, shown as a uniform layer in FIG. 34, could also be made in a variety of shapes and, in particular, could be made thinner near the outer heart wall and thicker near the inner heart wall. A thin-to-thick configuration would cause the hydrophilic layer to assume a more wedge-like form as water is absorbed, which would lock it even more securely in the hole formed in the heart.

It should also be appreciated that, although the hydrophilic layer is shown (for clarity) in FIGS. 33 and 34 with a particular embodiment of LV connector, the middle hydrophilic layer will work similarly with any LV connector embodiment herein disclosed.

### Thirteenth LV Connector Construction

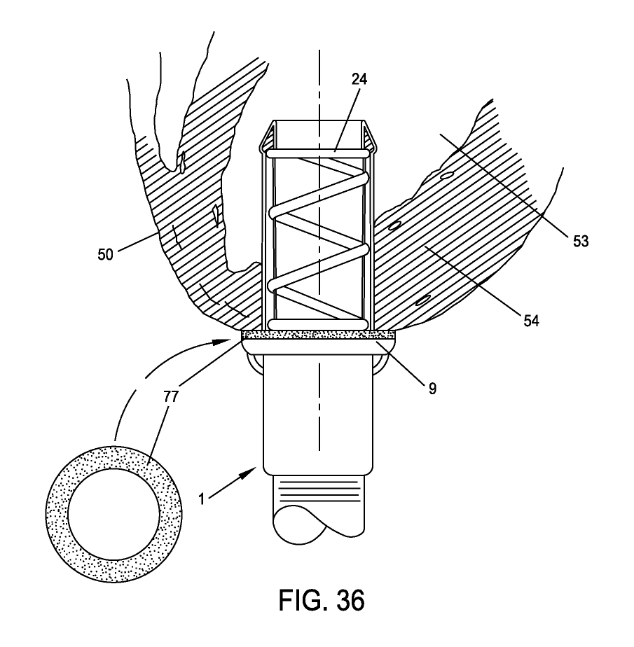

FIG. 36 shows another LV connector formed in accordance with the present invention. The LV connector shown in FIG. 36 is generally similar to the LV connector shown in FIGS. 3-9, except that in this form of the invention, there is provided, between flange 9 and heart wall 54, a layer, preferably in the shape of a ring or washer 77, of hydrophilic foam or material made, for example, of isocyanate-capped polyester pre-polymer or various compositions of a polyelectrolyte and polyvinyl alcohol, or various implantable hydrogels, especially bioresorbable hydrogels or keratin-type hydrogels used for tissue expansion. Upon implantation, as ring or washer 77 absorbs water from the body, ring or washer 77 expands so as to form a better seal between the LV connector and the heart.

Furthermore, if the hydrophilic washer 77 material is impregnated with, for example, collagen fibers, then the expanding hydrophilic layer 75 will also expedite the clotting cascade, thereby sealing any blood pathways that might exist around the tightly implanted LV connector.

It should be appreciated that, although the hydrophilic layer is shown (for clarity) in FIG. 36 with a particular embodiment of the LV connector, the hydrophilic washer 77 will work similarly with any LV connector embodiment herein disclosed.

### Fourteenth LV Connector Construction

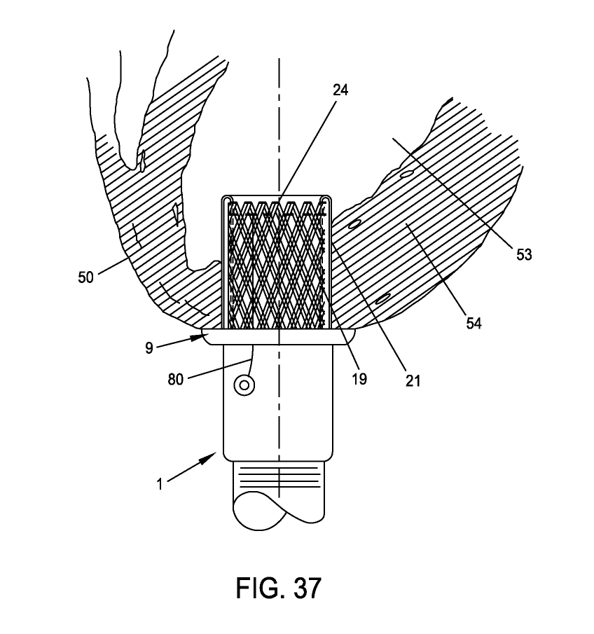

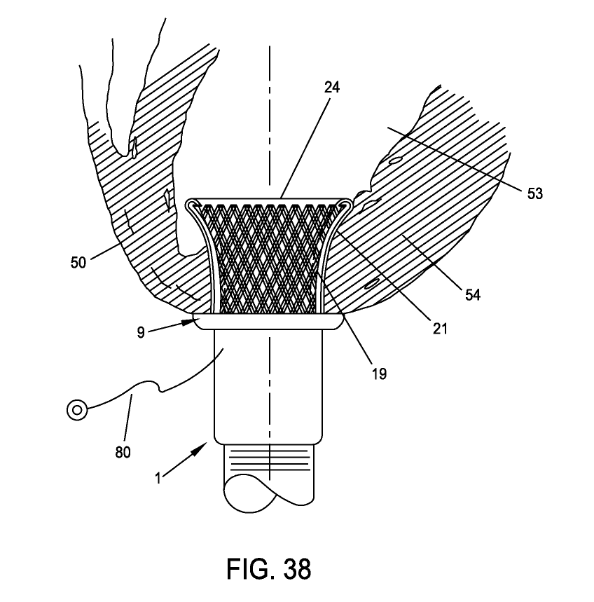

FIGS. 37 and 38 show another LV connector formed in accordance with the present invention. The LV connector shown in FIGS. 37 and 38 comprises a hollow expandable stent comprising an internal skeleton in the form of a frusto-conical structure 24. In this form of the invention, frusto-conical structure 24 comprises a series of interwoven wire torsional springs.

As seen in FIG. 37, the interwoven wire torsional springs may be forced into a torsionally-contracted (i.e., generally cylindrical) configuration, and then held in place by a stay suture and ripcord 80.

FIG. 38 shows frusto-conical structure 24 in its released condition, where the interwoven wire torsional springs resume their normal (i.e., unbiased) bell or trumpet shape. Alternately, or in addition to self-deployment resulting from the frusto-conical structure returning to its normal condition, suture or wire pulls (not shown) may be interwoven through the interwoven wire torsional springs in such a way that they exit through the flange where they may be tightened, and so pull and secure the interwoven wire torsional springs in the bell or trumpet shape seen in FIG. 38.

### Fifteenth LV Connector Construction

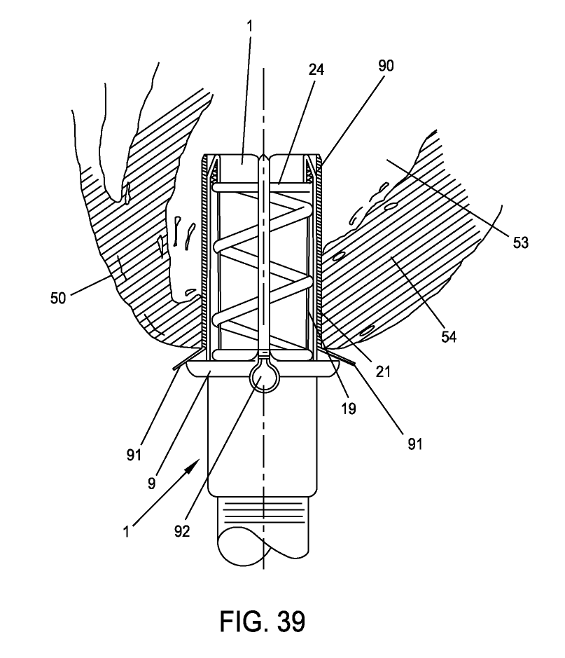

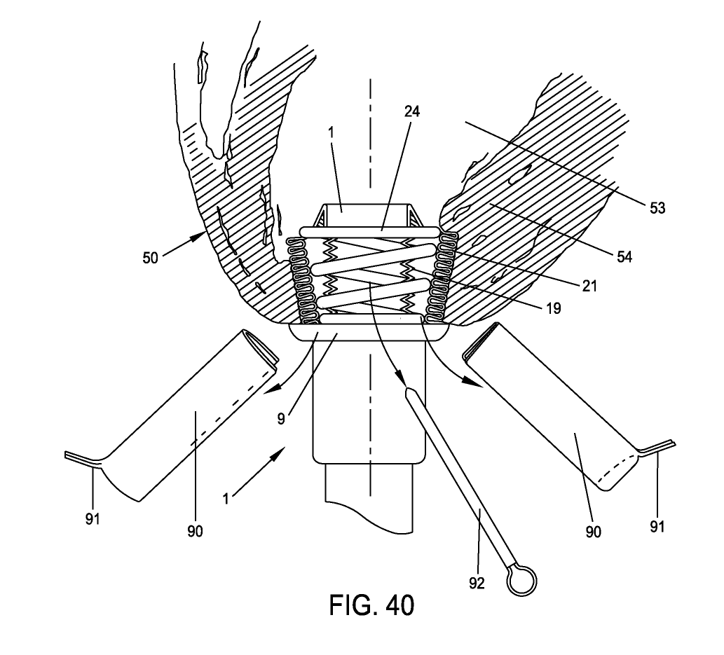

FIGS. 39 and 40 show another LV connector formed in accordance with the present invention. The LV connector shown in FIGS. 39 and 40 is generally similar to the LV connector shown in FIGS. 21 and 22, except that in this form of the invention, frusto-conical spring 24 is held in its diametrically-reduced state by a tubular element which may be removed when it is desired to transform the frusto-conical spring from its diametrically-reduced (i.e., generally cylindrical) state into its diametrically-expanded (i.e., generally frusto-conical) state.

More particularly, and looking now at FIG. 39, the LV connector may include a tubular element 90, preferably made of a thin or film-like plastic material, and containing stop element(s) 91 and tear strip element(s) 92. During implantation of the LV connector, the stop element 91 prevents flange 9 from making contact with the outer surface of the heart wall 54. The space maintained between the heart wall and the flange is provided in order to facilitate removal of the tear strip 92 and the tubular element(s) 90.

FIG. 40 shows the LV connector of FIG. 39 collapsing into its “final” (i.e., deployed) position. More particularly, tear strip 92 is pulled off and the tubular elements 90 are removed from around the LV connector, allowing frusto-conical spring 24 to axially contract and radially expand, thereby pulling membranes 19 and 21 into their folded or pleated conditions and pulling flange 9 into contact with the outer surface of the heart 54.

It should be appreciated that, although the removable tubular containment element 90 is shown (for clarity) in FIGS. 39 and 40 with a particular embodiment of the LV connector, it will work similarly with other LV connector embodiments herein disclosed.

### Sixteenth LV Connector Construction

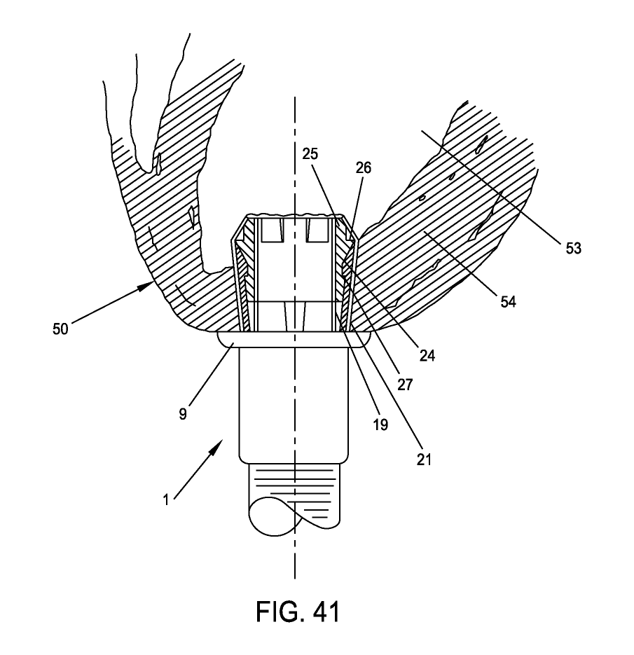

FIGS. 41-44 show another LV connector formed in accordance with the present invention. The LV connector shown in FIGS. 41-44 comprises a hollow expandable stent comprising an internal skeleton in the form of a frusto-conical structure 24, wherein the frusto-conical structure comprises a series of fingers arranged in a circle, fixed at one end at the flange 9 and free at the opposite (i.e., distal) end.

More particularly, and looking now at FIG. 41, the LV connector comprises a plurality of proximally-hinged fingers.

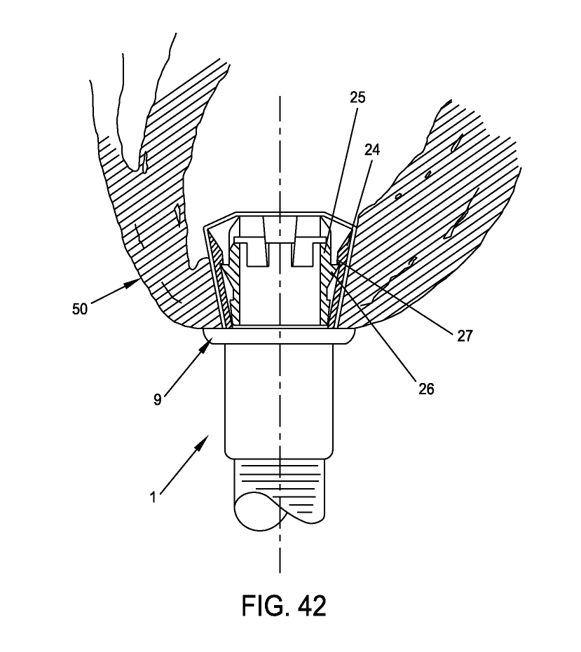

An expanding mechanism 25, preferably in the form of a ring-like element that rides on the inner surface of the fingers, is also provided. The inner surfaces of the fingers, and the outer surface of the expanding mechanism, have latching features 26 and 27. Inner membrane 19 and outer membrane 21 are shown in FIG. 42 in a vertically pleated or folded condition, which allows for radial expansion when the expanding mechanism forces the distal ends of the proximally-hinged fingers radially outward.

FIG. 42 shows that, as the expanding mechanism 25 is moved proximally toward flange 9, the proximally-hinged fingers are forced radially outward until the latching elements 26 and 27 engage, thereby locking frusto-conical structure 24 in a radially-expanded conical state. Outer membrane 21 and inner membrane 19 expand radially outwardly, losing their initial pleated configuration and conforming to the indentations between the opened fingers. The indented membrane surface allows for better engagement with the adjacent tissue surface while the expanded overall conical shape of frusto-conical structure 24, and the expanded conical membranes 19 and 21, serve to prevent the LV connector implant from coming out of the hole formed in the heart.

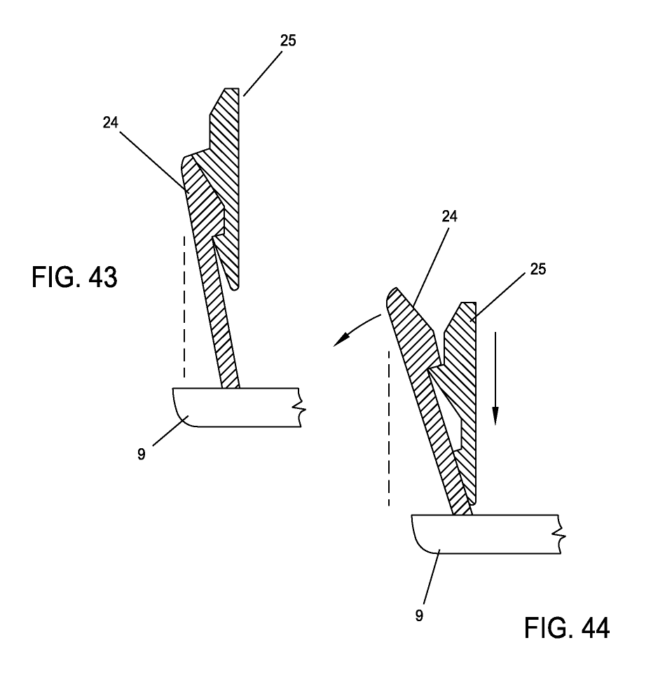

FIGS. 43 and 44 show schematic cross-sections of the latching mechanisms on the proximally-hinged fingers and expanding mechanism 25. More particularly, FIG. 43 shows the proximally-hinged fingers and the expanding mechanism 25 in an initial position, and FIG. 44 shows the proximally-hinged fingers and the expanding mechanism as the expanding mechanism moves distally along the surfaces of the proximally-hinged fingers, with the distal ends of the fingers moving outwardly until the latching elements 26 and 27 engage and lock the frusto-conical structure in a diametrically-expanded state. The actuating mechanism for moving expanding mechanism 25 could be, by way of example but not limitation, a pull wire, a pull pin, one or more sutures, or multiples of the same, and/or other tensioning means that pass through the flange and can be actuated from the outside the heart after the LV connector has been passed into the left ventricle.

### Seventeenth LV Connector Construction

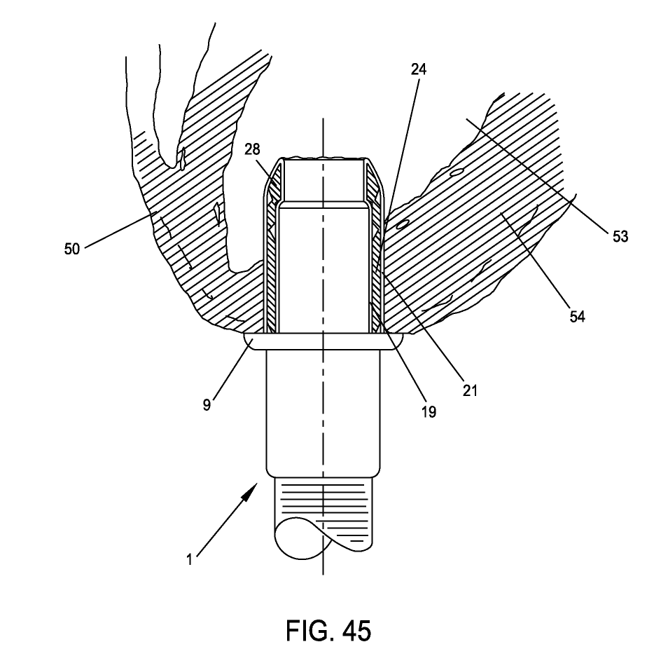

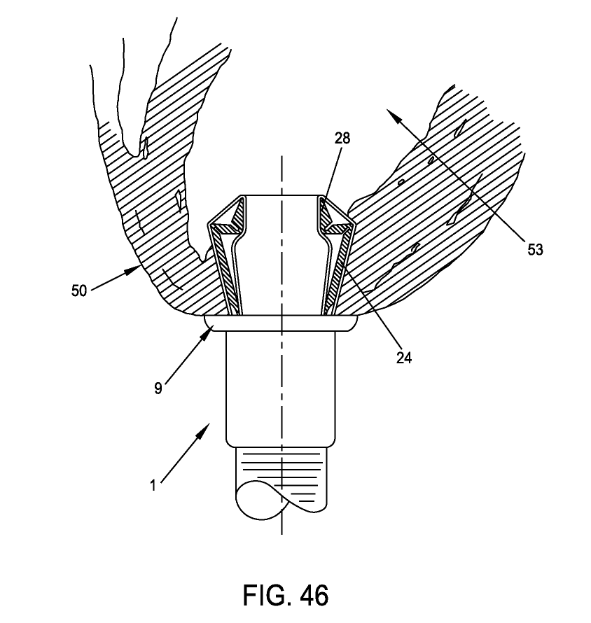

FIGS. 45 and 46 show another LV connector formed in accordance with the present invention. The LV connector shown in FIGS. 45 and 46 comprises a hollow expandable stent comprising an internal skeleton in the form of a frusto-conical structure 24, wherein the frusto-conical structure comprises a series of fingers arranged in a circle, hingeably fixed at their proximal ends near the flange, and hingeably joined at their distal ends to a ring-like element 28.

More particularly, and looking now at FIG. 45, the LV connector comprises a plurality of fingers proximally-hinged near the flange and distally-hinged to a ring-like element 28. Other hingable joints occur along the length of the fingers such that the fingers of the frusto-conical structure 24 are able to fold upon themselves while expanding radially outward in response to proximal movement of the ring-like element 28.

FIG. 46 shows the LV connector after ring-like element 28 has been displaced toward the flange. In response, the fingers of frusto-conical structure 24 bend at the hinge points, buckling radially outwardly. The actuating mechanism for producing the folding movement of frusto-conical structure 24 could be a pull wire, a pull pin, one or more sutures, or multiples of the same, or other tensioning means that pass through the flange and can be actuated from the outside of the heart after LV connector has been inserted into the left ventricle.

### Eighteenth LV Connector Construction

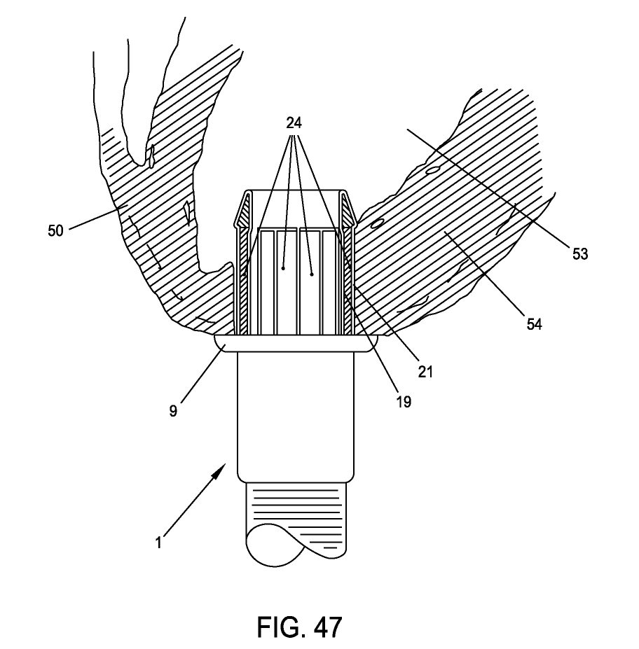

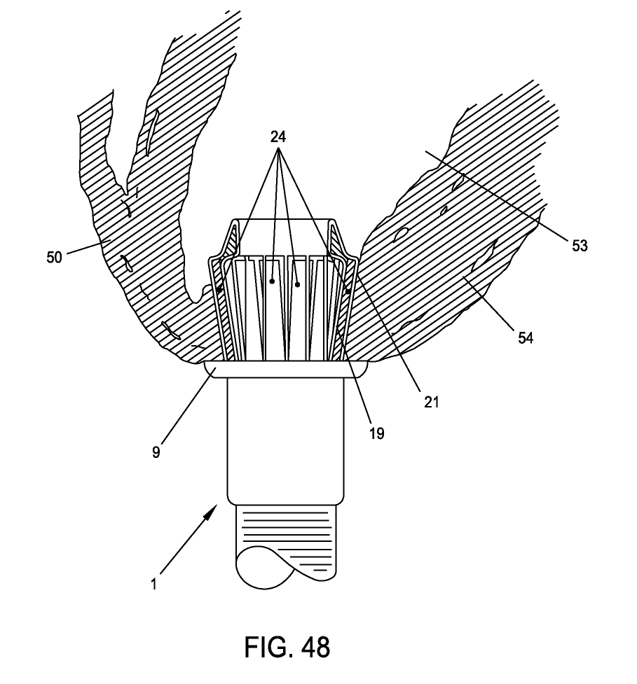

FIGS. 47 and 48 show another LV connector formed in accordance with the present invention. The LV connector shown in FIGS. 47 and 48 comprises a hollow expandable stent comprising an internal skeleton in the form of a frusto-conical structure 24, wherein the frusto-conical structure comprises a series of cantilevered fingers arranged in a circle, the fingers being attached at their flange ends and free at their distal ends. Between the finger spring elements, vertical support elements support the tapered distal end.

More particularly, and looking now at FIG. 47, the LV connector comprises a series of cantilevered fingers arranged into a circle, the fingers being attached near their flange ends and free at their distal ends. Between the finger spring elements, vertical support elements support the tapered distal ends. The fingers of frusto-conical structure 24, normally biased in a radially outward position, are maintained in a compressed position substantially parallel to the central axis of the LV connector. The compressing and holding means could be, for example, a releasable suture constricting the outer membrane 21. Alternatively, the restricting means could be a thin film tubular restrictor, with tear strip release, of the sort shown in FIG. 47.

FIG. 48 shows that when the restricting means is removed from the LV connector of FIG. 47, the fingers of frusto-conical structure 24 expand radially outwardly, whereupon the LV connector assumes a conical shape, pressing firmly against the tissue of the formed hole and maintaining the LV connector securely in the heart wall.

### MODIFICATIONS

While the invention has been described with particular reference to the preferred embodiments, it will be understood by those skilled in the art that various changes may be made and equivalents substituted for elements of the preferred embodiments without departing from the scope of the present invention. In addition, many modifications may be made to adapt a particular situation and material to a teaching of the present invention without departing from the essential teachings of the present invention.

1. An implantable connector for suturelessly connecting a conduit to a hollow organ, the implantable connector comprising:
a hollow expandable stent, wherein the hollow expandable stent comprises an internal skeleton and a blood-retaining membrane covering the internal skeleton, and wherein the hollow expandable stent is constructed so that it is capable of assuming (i) a diametrically-reduced state for insertion into an opening formed in the side wall of the hollow organ in order to create a first interference fit therewith, and (ii) a diametrically-expanded state for expanding against the side wall of the hollow organ in order to create a second, enhanced interference fit therewith.

2. An implantable connector according to claim 1 wherein the hollow expandable stent is constructed so that a change in the length of the internal skeleton results in a change in the diameter of the hollow expandable stent.

3-14. (canceled)

15. An implantable connector according to claim 2 wherein a change in the length of the internal skeleton results in a change in the diameter of the internal skeleton so as to cause a change in the diameter of the hollow expandable stent.

16. An implantable connector according to claim 1 wherein the hollow expandable stent is constructed so that (i) it has a generally cylindrical configuration when it is in its diametrically-reduced state, and (ii) it has a generally frusto-conical configuration when it is in its diametrically-expanded state.

17. An implantable connector according to claim 16 wherein the hollow expandable stent is constructed so that (i) it has a generally cylindrical configuration when it is in its diametrically-reduced state, and (ii) it has a generally frusto-conical configuration when it is in its diametrically-expanded state, with the wider end of the frusto-conical configuration being disposed distally of the narrower end of the frusto-conical configuration.

18. An implantable connector according to claim 16 wherein the hollow expandable stent is constructed so that (i) it has a generally cylindrical configuration when it is in its diametrically-reduced state, and (ii) it has a generally frusto-conical configuration when it is in its diametrically-expanded state, with the wider end of the frusto-conical configuration being disposed distally of the narrower end of the frusto-conical configuration, and further wherein a flange is formed on the hollow expandable stent proximal to the distal end of the hollow expandable stent.

19-20. (canceled)

21. An implantable connector according to claim 18 wherein a foam layer is secured to the blood-retaining membrane.

22. An implantable connector according to claim 21 wherein the foam layer is disposed external to the blood-retaining membrane.

23-41. (canceled)

42. An implantable connector according to claim 18 wherein the internal skeleton comprises a plurality of movable elements which provide the internal skeleton with its desired characteristics.

43. An implantable connector according to claim 42 wherein the internal skeleton comprises at least two telescoping members.

44. An implantable connector according to claim 43 wherein the internal skeleton comprises a latch mechanism for maintaining the at least two telescoping members in a selected configuration.

45. An implantable connector according to claim 44 wherein the selected configuration is a generally frusto-conical configuration.

46. (canceled)

47. An implantable connector according to claim 42 wherein the internal skeleton comprises a plurality of longitudinally extending members hinged along their length.

48. (canceled)

49. An implantable connector according to claim 42 wherein the internal skeleton comprises a plurality of cantilevered fingers.

50. (canceled)

51. A system for suturelessly connecting a conduit to a hollow organ, the system comprising:
an applicator comprising a pushing component, a coring component, and an expansion/retractor component, the coring component being mounted to the pushing component, and the expansion/retractor component being slidably coupled to the coring component and adapted to pass through a side wall of the hollow organ; and
an implantable connector mounted to the coring component of the applicator, the implantable connector comprising:
a hollow expandable stent, wherein the hollow expandable stent comprises an internal skeleton and a blood-retaining membrane covering the internal skeleton, and wherein the hollow expandable stent is constructed so that it is capable of assuming (i) a diametrically-reduced state closely sized to the coring component for insertion into an opening formed in the side wall of the hollow organ in order to create a first interference fit therewith, and (ii) a diametrically-expanded state substantially larger than the coring component for expanding against the side wall of the hollow organ in order to create a second, enhanced interference fit therewith.

52-54. (canceled)

55. A system according to claim 51 wherein the hollow expandable stent further comprises a first mount for releasable connection to a first corresponding mount on the applicator, and a second mount for releasable connection to a second corresponding mount on the applicator, the first mount on the hollow expandable stent being disposed adjacent to the distal end of the hollow expandable stent and the second mount on the hollow expandable stent being disposed proximal to the first mount.

56. A system according to claim 55 wherein the applicator is constructed so that the second corresponding mount on the applicator is movable relative to the first corresponding mount on the applicator.

57. A system according to claim 56 wherein the second corresponding mount on the applicator is axially movable relative to the first corresponding mount on the applicator.

58-85. (canceled)

86. A method for suturelessly connecting a conduit to a hollow organ, the method comprising the steps of:
mounting an implantable connector to a coring component;
forming an opening in the side wall of the hollow organ by advancing the coring component with respect to the side wall of the hollow organ, with the implantable connector being carried into the opening formed by the coring component;
diametrically expanding the implantable connector within the formed opening so as to secure the implantable connector to the side wall of the hollow organ, and removing the coring component from the formed opening.

87. A method according to claim 86 wherein the implantable connector comprises:
a hollow expandable stent, wherein the hollow expandable stent comprises an internal skeleton and a blood-retaining membrane covering the internal skeleton, and wherein the hollow expandable stent is constructed so that it is capable of assuming (i) a diametrically-reduced state closely sized to the coring component for insertion into an opening formed in the side wall of the hollow organ in order to create a first interference fit therewith, and (ii) a diametrically-expanded state substantially larger than the coring component for expanding against the side wall of the hollow organ in order to create a second, enhanced interference fit therewith.

88-118. (canceled)
